(19) United States
(12) Patent Application Publication
(10) Pub. No.: US 2026/0297020 A1
(43) Pub. Date: Oct. 1, 2026

(54) FREE-RADICALLY POLYMERISABLE COMPOUND AND COMPOSITION

(71) Applicant: Mühlbauer Technology GmbH, Hamburg (DE)

(72) Inventors: Stephan Neffgen, Pinneberg (DE); Henrik Böttcher, Tostedt (DE); Swen Neander, Hamburg (DE)

(21) Appl. No.: 19/480,628

(22) PCT Filed: Mar. 12, 2024
(86) PCT No.: PCT/EP2024/056523

§ 371 Date: Oct. 31, 2025

(30) Foreign Application Priority Data
May 2, 2023 (DE) 102023111186.8

(51) Int. Cl. C07C 271/24; A61C 5/77; A61C 13/00; A61K 6/16; A61K 6/54; A61K 6/62; A61K 6/77; A61K 6/79; A61K 6/836; A61K 6/887; B33Y 70/10; B33Y 80/00; C08F 2/50; C08F 122/10; C08K 5/00

(52) U.S. Cl. CPC C07C 271/24; A61C 13/0019; A61K 6/54; A61K 6/79; A61K 6/887; B33Y 70/10; B33Y 80/00; C08F 2/50; C08F 122/1006; C08K 5/005; A61C 5/77; A61K 6/16; A61K 6/62; A61K 6/77; A61K 6/836; C07C 2603/12

(57) ABSTRACT

The invention relates to a radically polymerisable compound represented by a structure of formula 1: PG-Sp-PCA-Sp-[OG (O) NH—K—NHC (O) O-Sp-PCA-Sp]ₙ-PG (formula 1), wherein PG=in each case independently selected from a radically polymerisable group, R¹=selected from hydrogen, a C1-C8 alkyl group, an aryl group and an araliphatic group having C6-C8 carbon atoms, R²=selected from hydrogen, a C1-C4 alkyl group; Sp=in each case independently of one another a spacer group selected from unbranched and branched alkylene with C1-C19 carbon atoms, which may additionally contain oxygen, sulfur and/or —OOC— in the carbon chain, or Sp is absent; PCA=in each case independently of one another a polycyclic group; K=an aliphatic acyclic, saturated or unsaturated unit having C1-C15 carbon atoms, an aliphatic cyclic, saturated or unsaturated unit having C3-C15 carbon atoms, or an aromatic or araliphatic unit having C6-C14 carbon atoms; n=1 to 9. The invention also relates to a process for preparing such a compound, a radically polymerisable composition containing such a compound, the use thereof and a cured dental material.

The invention relates to a free-radically polymerizable compound, to a process for preparing such a compound, to a free-radically polymerizable composition comprising such a compound, to the use of these, and to a hardened dental material.

Free-radically polymerizable dental materials contain primarily (meth)acrylate monomers. Dimethacrylate systems are usually used for restorative and prosthetic dental materials such as dental fillings or dentures, on account of their properties such as rapid free-radical polymerization, good mechanical properties and esthetic appearance. Customary monomers are, for example, high molecular weight linear structures containing aliphatic or aromatic groups and having terminal methacrylate functionalities, such as 2,2-bis[4-(2-hydroxy-3-methacryloyloxypropoxy)phenyl]propane (bis-GMA) and 7,7,9-trimethyl-4,13-dioxo-3,14-dioxa-5,12-diazahexadecane-1,16-diyl bis(2-methylacrylate) (UDMA).

There have for some time been efforts to very substantially cease using bis-GMA or other monomers having bisphenol A-derived structural elements, and to replace them with other compounds at least to some degree. The spotlight here has been on urethane monomers and oligomers in particular. In the field of dental materials, the substance with most widespread commercial use as an at least partial substitute for bis-GMA is UDMA.

Monomers such as bis-GMA and UDMA, although present in wide ranges of commercial free-radically polymerizable composition for production of dental materials, have some drawbacks. They are generally highly viscous to solid substances. Mixtures with monomers having significantly low viscosity, such as triethylene glycol dimethacrylate (TEDMA), are accordingly used. TEDMA is a very versatile, low molecular weight monomer with low viscosity (of 0.01 Pa·s at 23° C.) and has high mobility during polymerization, which facilitates conversion in the polymerization.

However, the polymerizable compositions comprising these monomer mixtures and the dental materials obtained therefrom have some problematic properties that can adversely affect clinical treatment outcomes. For example, compositions comprising dimethacrylate monomers have relatively low polymerization conversions, significant polymerization shrinkage, poor toughness and unwanted water absorption. The known systems are often able to achieve only a comparatively low conversion of the double bonds, which not only contributes to inadequate mechanical properties and poor wear resistance, but is also disadvantageous in respect of the toxicology and biocompatibility of the polymerized dental materials. In addition, the volume shrinkage of the currently used dimethacrylate monomers and the shrinkage stresses of a tooth filling can result in failure of the bond between tooth and filling, leading to microleaks and consequently to secondary caries, which in turn can considerably reduce the longevity of the restoration. Attempts to boost the double bond conversion so as to reduce the level of unconverted monomers regrettably lead to an increase in polymerization shrinkage and shrinkage stress.

Low molecular weight monomers having oligo[ethyleneoxy] groups, such as TEDMA, which have a degree of solubility in water and thus bioavailability, are now being assessed critically on account of their toxicological properties and susceptibility to biodegradation processes. Monomers containing the bis-2,2[p-oxyphenyl]propane structural element, i.e. monomers based on bisphenol A, are likewise being assessed critically, since dental materials comprising monomers or oligomers having these structural elements have been found to release detectable amounts of bisphenol A, to which toxicologically critical properties are attributed.

There are various approaches to increasing conversion or reducing volume shrinkage. In dental composites for dental fillings, which comprise filler in a matrix of organic resin, attempts are being made to reduce volume shrinkage by increasing the filler content. However, if the filler content is too high, it is difficult to mix the fillers with the organic resin. In addition, the filler content is limited for dental composites, which must have a certain fluidity. For increased conversion and reduced polymerization shrinkage, there is ongoing development of novel monomers, for example high molecular weight urethane methacrylate monomers. The synthesis of these monomers is complex and typically also requires purification steps, which leads to a limited availability of such monomers. Increasing the molecular weight is generally associated with poorer mechanical properties in the cured dental materials for a given monomer functionality. Moreover, the elevated viscosity of such monomers means they have to be used alongside higher amounts of low-viscosity monomers in order to permit use for dental composites, which has an adverse effect on shrinkage.

EP 2436365 B1 describes low-shrinkage dental composites comprising monomer mixtures containing monomers (b1) and (b2) in a ratio of 1:20 to 5:1. The example compositions each contain 4.8-76.6% by weight of bis((meth)acryloyloxymethyl)tricyclo[5.2.1.0^{2,6}]decane (b1), 90.9-19.1% by weight of UDMA (b2), and 4.3% by weight of TEDMA (b2). These composites display a polymerization shrinkage of about 1.50% irrespective of the ratio of (b1) to (b2). When, as in comparative example 11, the filler content is reduced and the proportion of TEDMA is increased, there is an increase in polymerization shrinkage.

Vaidyanathan et al. “Visible light cure characteristics of a cycloaliphatic polyester dimethacrylate alternative oligomer to bis-GMA”, Acta Biomater Odontol Scand. 2015; 1:59-65, disclose the use of PEM-665 as a BPA-free alternative to bis-GMA in combination with 30% or 50% by weight of TEDMA. Investigation of the polymerization conversions of these mixtures found that the combinations of PEM with TEDMA showed a higher percentage polymerization conversion than the combinations of bis-GMA with TEDMA.

There is still a need for free-radically polymerizable compounds or free-radically polymerizable compositions containing such compounds that can enable a reduced toxicity potential and reduced volume shrinkage with simultaneously good mechanical properties of the dental material to be produced therefrom and that are readily available.

It is therefore an object of the present invention to provide a free-radically polymerizable compound or a free-radically polymerizable composition containing such a compound that overcomes the above-detailed disadvantages of the prior art. In particular, it is to be made possible to provide a free-radically polymerizable compound and a free-radically polymerizable composition with which it is possible to obtain dental materials with reduced polymerization shrinkage with simultaneously good mechanical properties of the dental material, such as very good flexural strength and tensile strength, and also good fracture toughness, and which are at the same time toxicologically safe. Furthermore, it is to be made possible to obtain a free-radically polymerizable compound or a free-radically polymerized composition containing such a compound that have a refractive index particularly suitable for production of materials that contain fillers customary in the dental sector and have advantageous optical properties, in particular with regard to translucence and opacity. Furthermore, it is an object of the present invention to provide a process that enables production of such a free-radically polymerizable compound in a simple manner, in high purity and without unwanted discoloration.

This object is achieved by the invention by means of a free-radically polymerizable compound, represented by a structure of the formula 1:

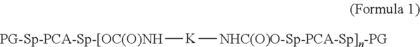

where
PG=each independently selected from a free-radically polymerizable group, preferably selected from (NR¹)OC—CR²═CH₂ and OOC—CR²═CH₂, more preferably selected from OOC—CR²═CH₂,
R¹=selected from hydrogen, a C1-C8-alkyl group, an aryl group and an araliphatic group with C6-C8 carbon atoms, preferably selected from a C1-C8-alkyl group and a benzyl group,
R²=selected from hydrogen, a C1-C4-alkyl group, preferably selected from hydrogen and methyl;
Sp=a spacer group selected independently from unbranched and branched alkylene having C1-C19 carbon atoms, which may additionally contain oxygen, sulfur and/or —OOC— in the carbon chain,
   or Sp is absent;
PCA=each independently a polycyclic group, preferably an aliphatic polycyclic group, preferably an aliphatic bi- or tricyclic group;
K=an aliphatic acyclic, saturated or unsaturated unit having C1-C15 carbon atoms, preferably C3-C13 carbon atoms, more preferably C6-C9 carbon atoms, where the unit may be substituted by one or more C1-C3 aliphatic carbon substituents,
   an aliphatic cyclic, saturated or unsaturated unit having C3-C15 carbon atoms, preferably C5-C13 carbon atoms, more preferably C6-C13 carbon atoms, where the unit may be substituted by one or more aliphatic C1-C3 carbon substituents, or
   an aromatic or araliphatic unit having C6-C14 carbon atoms, preferably C6-C13 carbon atoms, where the unit may be substituted by one or more aliphatic C1-C3 carbon substituents;
n=1 to 9, preferably 1 to 6.

Preferred embodiments can be found in the subclaims.

First of all, some terms used in the context of the invention will be explained.

What is meant in the context of the present invention by (polymerizable) dental materials is materials for (bio)medical use, in particular on hard tooth substance, such as enamel and dentine, or on bone tissue, such as on the jawbone.

In the context of the present invention, the free-radically polymerizable compounds are monomers if n=1, and oligomers if n=2 to 9.

Preferably, K is selected from a linear aliphatic saturated unit having C6-C9 carbon atoms, where the unit may be substituted by one or more aliphatic C1-C3 carbon substituents, aliphatic cyclic saturated unit having C6-C13 carbon atoms, where the unit may be substituted by one or more aliphatic C1-C3 carbon substituents, and an aromatic or araliphatic unit having C6-C13 carbon atoms having at least two aliphatic substituents, preferably C1-C3 substituents, on the aromatic ring.

Furthermore, K may be 1,3- and 1,4-cyclohexanylene, preferably 1,3-cyclohexanylene.

The spacer group Sp is preferably selected from methylene, *CH₂—(OC₂H₄)ₚ, *CH₂—(OC₃H₆)ₚ, *(OC₂H₄)ₚ, *(OC₃H₆)ₚ, *CH₂—(O—C(O)—R⁵—)ₚ, *(O—C(O)—R⁵—)ₚ and *S—R⁵, where p=1-5, preferably 1-3, and R⁵ is a C1-C12 alkylene group, preferably a C2-C6 alkylene group. The symbol * indicates the bonding site to the PCA unit.

The spacer group Sp is more preferably methylene.

The polycyclic group PCA is preferably in each case selected from structures of the following formulae 2 to 13:

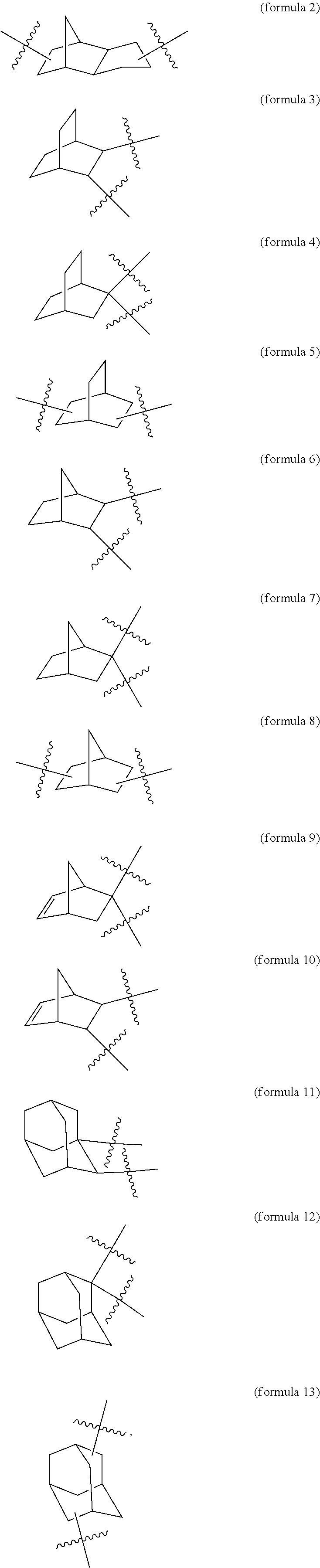

which may optionally be substituted by one or more C1-C4 alkyl groups. Thus also included in each case are all stereoisomers of these formulae, in particular enantiomers and diastereomers.

Furthermore, the polycyclic group PCA is preferably selected from 2-methyl-3,3-norbornanediyl, 2-ethyl-3,3-norbornanediyl, 2-propyl-3,3-norbornanediyl, 2-butyl-3,3-norbornanediyl, bicyclo[2.2.2]octane-1,4-diyl, bicyclo[2.2.2]octane-2,3-diyl, bicyclo[2.2.2]oct-5-ene-5,6-diyl, 1,3-adamantanediyl, tetracyclo[6.6.2.0^{2,7}.0^{9,14}]hexadeca-2,4,6,9,11,13-hexaene-15,16-diyl, 1,4-cubanediyl, 2,6,6-trimethylbicyclo[3.1.1]heptane-2,3-diyl and 1,7,7-trimethylbicyclo[3.1.1]heptane-2,3-diyl. In a preferred embodiment, the polycyclic group PCA is an aliphatic tricyclic group, more preferably tricyclo[5.2.1.0/2.6]decanylene (TCD) (formula 2).

K is preferably selected from the structures of the following formulae 14 to 26:

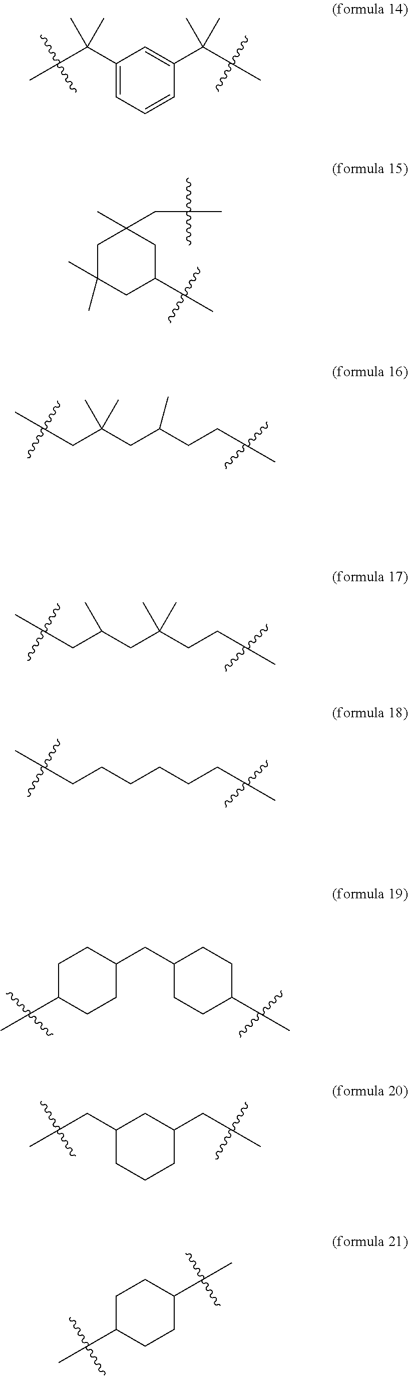

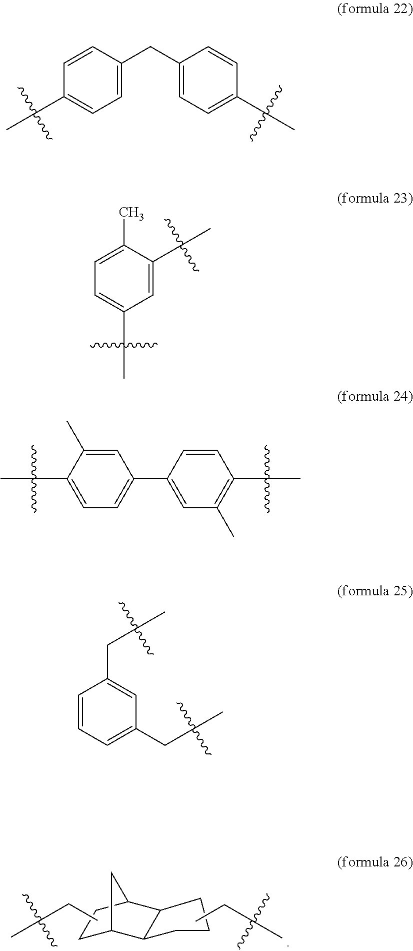

Furthermore, K is preferably selected from the following groups: 1,5-naphthylene (from naphthylene 1,5-disocyanate), 2,4,6-triisopropyl-m-phenylene (from 2,4,6-triisopropyl-m-phenylene diisocyanate), 2,5-bis(methanediylbicyclo[2.2.1]heptane and 2,6-bis(methanediyl)bicyclo[2.2.1]heptane (from norbornane-2,5-diylbis(methylene)diisocyanate or norbornane-2,6-diylbis(methylene)diisocyanate).

K is more preferably selected from a structure of the formulae 14, 15, 16, 17, 18, 19 and 20.

In one embodiment, the free-radically polymerizable compound is preferably represented by a structure of the formula 27:

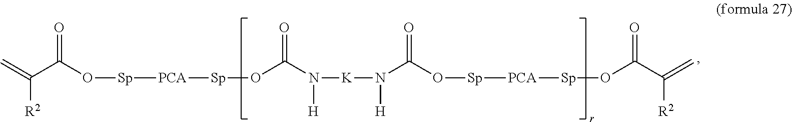

where
R²=hydrogen or a methyl group;
Sp=a spacer group selected independently from unbranched and branched alkylene having C1-C19 carbon atoms, which may additionally contain oxygen, sulfur and/or —OOC— in the carbon chain,
   or Sp is absent;
PCA=each independently a polycyclic group, preferably an aliphatic polycyclic group, preferably an aliphatic bi- or tricyclic group, more preferably a tricyclic group, most preferably tricyclo[5.2.1.0/2,6]decanylene;
K=an aliphatic acyclic, saturated or unsaturated unit having C1-C15 carbon atoms, preferably C3-C13 carbon atoms, more preferably C6-C9 carbon atoms, where the unit may be substituted by one or more C1-C3 aliphatic carbon substituents,
   an aliphatic cyclic, saturated or unsaturated unit having C3-C15 carbon atoms, preferably C5-C13 carbon atoms, more preferably C6-C13 carbon atoms, where the unit may be substituted by one or more aliphatic C1-C3 carbon substituents, or
   an aromatic or araliphatic unit having C6-C14 carbon atoms, preferably C6-C13 carbon atoms, where the unit may be substituted by one or more aliphatic C1-C3 carbon substituents;
r=1 to 9, preferably 1 to 6.

The free-radically polymerizable compound is preferably represented by a structure selected from the following formulae 28-31:

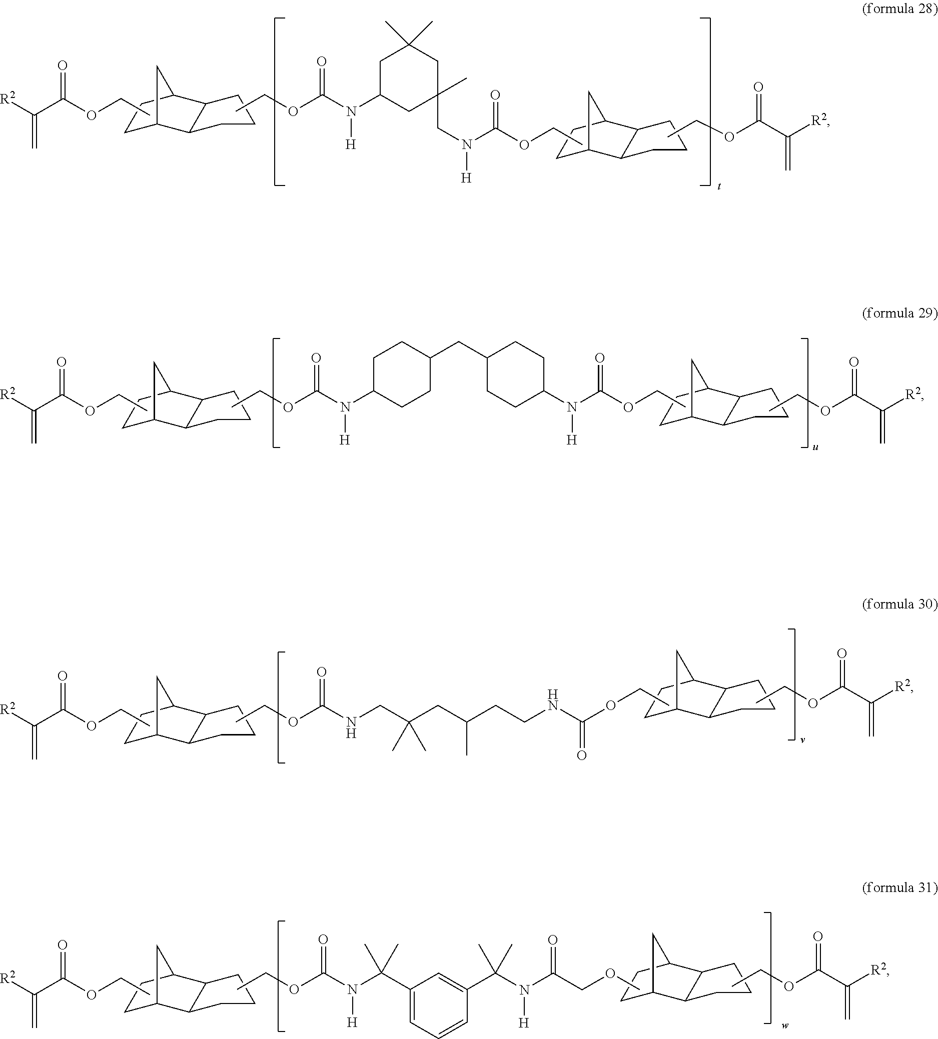

where
t, u, v and w=each independently 1-9, preferably 1-6; and
R²=selected from hydrogen, a C1-C4 alkyl group, preferably selected from hydrogen and a methyl group.

In the case of the free-radically polymerizable compound represented by a structure selected from the following formulae 28-31, it should be noted that a respective reverse direction of the tricyclo[5.2.1.0/2,6]decanylene groups is included.

The invention further provides a process for preparing a free-radically polymerizable compound, characterized in that the process comprises the following steps:
a) reacting a diol of a polycyclic group PCA with a (meth)acrylic ester to give a PCA mono(meth)acrylate having a hydroxyl group by enzymatic catalysis,
b) reacting the PCA mono(meth)acrylate containing the hydroxyl group from step a) with a diisocyanate compound, preferably using a catalyst.

The PCA mono(meth)acrylate reaction product with a hydroxyl group from step a) can be prepared by transesterification from PCA di(meth)acrylate and PCA diol, preferably TCD di(meth)acrylate (TCD D(M)A) and TCD dimethanol. The corresponding mono(meth)acrylate is preferably formed in each case with one (meth)acrylate group and one OH group in a mixture with PCA D(M)A and residues of PCA dimethanol. Surprisingly, low-color to colorless reaction products are producible by this reaction step, and these are distinctly different from compounds prepared by acid catalysis, for example, which are usually intensely colored. In the case of the latter compounds, a more complex cleaning procedure is generally necessary prior to use, at least in fields where appearance is also important.

The reaction (transesterification) in step a) is effected by enzymatic catalysis. Preferred enzymes are lipases or mixtures of lipases. In a preferred embodiment, CALB is used. In a particularly preferred embodiment, CALB is used immobilized on a carrier.

The diol from step a) is preferably selected from primary and secondary alcohols containing a polycyclic group PCA. Suitable primary diols may be bis(hydroxymethyl)tricyclo[5.2.1.0^{2,6}]decane (isomer mixture), 5-norbornene-2,2-dimethanol, 5-norbornene-2,3-dimethanol, bicyclo[2.2.1]heptane-2,3-dimethanol, 2-methyl-3,3-norbornanedimethanol, 2-ethyl-3,3-norbornanedimethanol, 2-propyl-3,3-norbornanedimethanol, 2-butyl-3,3-norbornanedimethanol, bicyclo[2.2.2]octane-1,4-dimethanol, bicyclo[2.2.2]oct-5-ene-5,6-diyldimethanol, bicyclo[2.2.2]octane-2,3-dimethanol, tricyclo[3.3.1.1^{3,7}]decane-1,3-diethanol, 1,3-adamantanedimethanol, pentacyclopentadecanedimethanol, tetracyclo[6.6.2.0^{2,7}.0^{9,14}]hexadeca-2,4,6,9,11,13-hexaene-15,16-diyldimethanol, 1,4-bis(hydroxymethyl)cubane and [5-(hydroxymethyl)-5,6-dimethyl-6-bicyclo[2.2.1]hept-2-enyl]methanol. Suitable secondary diols may be bicyclo[2.2.1]hept-2-ene-1,2-diol, bicyclo[2.2.1]heptane-1,2-diol, bicyclo[2.2.1]heptane-2,5-diol, bicyclo[2.2.1]heptane-1,4-diol, 2,6,6-trimethylbicyclo(3.1.1)heptane-2,3-diol, 1,7,7-trimethylbicyclo[2.2.1]heptane-2,3-diol, 2,3-dihydroxynorbornane, 2-(propyl-1,2-diol)norbornane, tricyclo[5.2.1.0^{2,6}]decane-3,4-diol and 2,6-dihydroxyadamantane. The diols are generally commercially available.

The (meth)acrylic ester from step a) is preferably selected from alkyl esters, vinyl esters, aryl esters and further active esters. It is possible to use both mono(meth)acrylates and di(meth)acrylates. In the case of mono(meth)acrylates, it is preferable that the resulting monoalcohol is removed from the mixture during the reaction. In the case of di(meth)acrylates, the use of the di(meth)acryloyl ester of PCA diol is preferred. What is formed in this case is the mono(meth)acryloyl ester of the PCA diol in a mixture with the diol and the di(meth)acryloyl ester of PCA diol. It is particularly advantageous that this mixture is of particularly low color and contains a high proportion of mono(meth)acrylate, which is favorable for the subsequent reaction in step b).

A catalyst can be used for step b). Suitable catalysts in step b) are preferably urethanization catalysts. Such catalysts accelerate the reaction rate of the reaction between a hydroxyl group and an isocyanate group. Examples of the urethanization catalyst include organotin compounds such as dimethyltin dineodecanoate, dibutyltin dilaurate, dibutyltin diocate and tin octoate; organobismuth compounds such as bismuth neodecanoate; organic compounds of metals other than tin, such as copper naphthenate, cobalt naphthenate, zinc naphthenate, acetylacetonatozirconium, acetylacelacetonatoiron and acetylacetonatogermanium; amine compounds and salts thereof, such as triethylamine, 1,4-diazabicyclo[2.2.2]octane, 1,8-diazabicyclo[5.4.0]undecene, N,N-dimethylcyclohexylamine, pyridine, N-methylmorpholine, N,N,N′,N′-tetramethylethylenediamine, N,N,N′,N′-tetramethyl-1,3-butanediamine, N,N,N′,N′-pentamethyldiethylenetriamine, N,N,N′,N′-tetra(340 dimethylaminopropyl)methanediamine, N,N′-dimethylpiperazine and 1,2-dimethylimidazole; and trialkylphosphine compounds such as tri-n-butylphosphine, tri-n-hexylphosphine, tricyclohexylphosphine and tri-n-octylphosphine. Preference is given here to dibutyltin dilaurate, dimethyltin dineodecanoate and bismuth neodecanoate.

The diisocyanate compound used in step b) is preferably selected from diisocyanates containing aliphatic, cycloaliphatic, polycyclic, araliphatic or aromatic structural elements. Suitable diisocyanate compounds may be 4,4′-methylenediphenyl diisocyanate (MDI), 4-methyl-m-phenylene diisocyanate and other isomers (TDI), 1,5-naphthylene diisocyanate (NDI), 1,3-bis(isocyanatomethyl)benzene (XDI), 1,3-bis(1-isocyanato-1-methylethyl)benzene (TMXDI), 2,4,6-triisopropyl-m-phenylene diisocyanate (TRIDI), 3,3′-dimethylbiphenyl-4,4′-diyl diisocyanate (TODI), 1,4-phenylene diisocyanate, hexamethylene diisocyanate (HDI), 2,2,4- and 2,4,4-trimethylhexamethylene 1,6-diisocyanate (TMDI), 3-isocyanatomethyl-3,5,5-trimethylcyclohexyl isocyanate and other isomers (IPDI), 4,4′-methylenedicyclohexyl diisocyanate (H12MDI), 2,5- and 2,6-bis(isocyanatomethyl)bicyclo[2.2.1]heptane (NBDI), 1,4-cyclohexane diisocyanate, 1,3-bis(isocyanatomethyl)cyclohexane (BIMC) and octahydro-2,5-bis(isocyanatomethyl)-4,7-methano-1H-indene (TCDDI). In general, the diisocyanate compounds are commercially available.

A ratio of a number of moles of the hydroxyl group from the PCA mono(meth)acrylate to a number of moles of the isocyanate group from the diisocyanate compound is preferably 0.5 to 1.5, more preferably 0.8 to 1.2. Even more preferably, this ratio is 1.0. A ratio of a molar amount of all hydroxyl groups to a molar amount of all isocyanate groups in the reaction in step b) is preferably 1.1 to 1.0, more preferably 1.02 to 1.0.

Conversions or reactions in steps a) and b) can be conducted in an inert solvent or solvent mixture or else, as the case may be, without solvent.

Solvents used for step a) may be a wide variety of solvents, provided that they are an inert solvent for the reaction. Examples include non-polar hydrocarbon-based solvents such as n-hexane, benzene, toluene and xylene; non-polar halogen-based solvents such as dichloromethane, chloroform, carbon tetrachloride, 1,2-dichloroethane and perchloroethylene; mid-polarity ketone-based solvents such as acetone, methyl ethyl ketone and methyl isobutyl ketone; mid-polarity ether-based solvents such as diethyl ether, diisopropyl ether, dibutyl ether, methyl tert-butyl ether, tetrahydrofuran and dioxane; and polar solvents such as acetonitrile, tert-butyl alcohol, N,N-dimethylformamide, N,N-dimethylacetamide, N,N-dimethylimidazolidinone, dimethyl sulfoxide and sulfolane. These solvents may be used individually or as a mixture. Nonpolar and mid-polarity solvents are preferred.

Solvents used for step b) may be a wide variety of solvents, provided that they are an inert solvent for the reaction. Examples include hydrocarbon-based solvents such as n-hexane, benzene, toluene and xylene; ketone-based solvents such as acetone, methyl ethyl ketone and methyl isobutyl ketone; ester-based solvents such as ethyl acetate and butyl acetate; ether-based solvents such as diethyl ether, diisopropyl ether, dibutyl ether, methyl tert-butyl ether, tetrahydrofuran and dioxane; halogen-based solvents such as dichloromethane, chloroform, carbon tetrachloride, 1,2-dichloroethane and perchloroethylene; and polar solvents such as acetonitrile, N,N-dimethylformamide, N,N-dimethylacetamide, N,N-dimethylimidazolidinone, dimethyl sulfoxide and sulfolane. These solvents may be used individually or as a mixture.

The reactions in steps a) and b) can be conducted within a temperature range from 0° C. to 100° C. The temperature range between 20® C. and 80° C. is preferred, further preferably between 40° C. and 60° C.

The reactions in steps a) and b) can be conducted in the presence of polymerization inhibitors. Polymerization inhibitors prevent the free-radical polymerization of the (meth)acrylate groups shown. Examples of polymerization inhibitors are 2,6-di-tert-butyl-4-methylphenol (BHT), hydroquinone (HQ), hydroquinone monomethyl ether (MEHQ), 2,2,6,6-tetramethylpiperidinyloxyl (TEMPO) and phenothiazine (PTZ).

After step b), it may be necessary under some circumstances, in a further step c), to remove solvent still present from the reaction mixture obtained.

The present invention further provides a free-radically polymerizable composition comprising:
a) one or more free-radically polymerizable compounds of the invention, preferably as claimed in any of claims 1 to 7;
b) optionally one or more further free-radically polymerizable monomers and/or oligomers not covered by the at least one free-radically polymerizable compound of the formula 1;
c) optionally at least one initiator or initiator system for the polymerization;
d) optionally one or more stabilizers;
e) optionally one or more fillers;
f) optionally customary dental additives.

It is preferable that, in the free-radically polymerizable composition, one or more free-radically polymerizable compounds of the formula 1 are present in a proportion by mass of 1-100% by weight, preferably 2-80% by weight, preferably 2-65% by weight, more preferably 10-65% by weight, based on the total mass of all free-radically polymerizable monomers and oligomers in the polymerizable composition.

The free-radically polymerizable composition preferably includes several compounds of the formula 1, selected from monomers (n=1) and/or oligomers (n=2-9, preferably n=2-5).

One or more of the following constituents may be present in the free-radically polymerizable composition in the following proportions by mass in each case, based on the total mass of the polymerizable composition:
a) one or more free-radically polymerizable compounds of the formula 1: from 1% to 99% by weight, preferably from 2% to 95% by weight, more preferably from 2% to 80% by weight, still more preferably from 2% to 65% by weight, still more preferably 10-65% by weight;
b) one or more other free-radically polymerizable monomers or oligomers not covered by the at least one compound of the formula 1: from 0% to 99% by weight, more preferably from 10% to 99% by weight, more preferably from 20% to 98% by weight, even more preferably from 35% to 90% by weight;
c) the at least one initiator or initiator system for the polymerization: from 0% to 5% by weight, preferably from 0.01% to 5% by weight;
d) the stabilizers: from 0% to 5% by weight, preferably from 0.001% to 5% by weight, more preferably 0.005-2% by weight;
e) the fillers or filler particles: from 0% to 95% by weight, preferably from 1% to 95% by weight, further preferably from 5% to 92% by weight;
f) the customary dental additives: from 0% to 5% by weight, preferably from 0.001% to 5% by weight.

In a preferred embodiment, the free-radically polymerizable composition is a free-radically polymerizable dental material.

### b) Further Polymerizable Monomers or Oligomers

The free-radically polymerizable composition may, in addition to the at least one free-radically polymerizable compound of the formula 1, also contain one or more further free-radically polymerizable monomers or oligomers that are not covered by the at least one free-radically polymerizable compound of the formula 1.

The selection of these further free-radically polymerizable monomers and oligomers is preferably made depending on which material is to be produced. This also applies to the proportion of the further free-radically polymerizable monomers and/or oligomers in the free-radically polymerizable composition.

Suitable further free-radically polymerizable monomers are selected, for example, from bis(methacryloyloxymethyl)tricyclo[5.2.1.0/2,6]decane, bis(acryloyloxymethyl)tricyclo[5.2.1.0/2,6]decane. The monomers may also be those obtainable by esterification reaction, for example according to the production examples of EP 0235836 B1 or U.S. Pat. No. 4,131,729/DE 2816823.

Suitable further free-radically polymerizable monomers may be selected from urethane (meth)acrylates having two or more (meth)acrylate groups. These are preferably urethane di(meth)acrylates and/or urethane tri(meth)acrylates. Urethane (meth)acrylates are preferably selected from linear or branched alkylene-functionalized urethane (meth)acrylates and urethane (meth)acrylate-functionalized polyethers.

Preference is given to difunctional urethane(meth)acrylates selected from difunctional urethane(meth)acrylates having a divalent alkylene group and those having a divalent cyclic aliphatic hydrocarbon group. Such difunctional urethane(meth)acrylates having a divalent alkylene group are preferably selected from linear or branched urethane di(meth)acrylates functionalized with a divalent alkylene group, urethane di(meth)acrylate-functionalized polyethers having alkylene group(s), such as bis(methacryloyloxy-2-ethoxycarbonylamino)alkylene, bis(methacryloyloxy-2-ethoxycarbonylamino)-substituted polyalkylene ethers. Preference is given to bis(methacryloyloxy-2-ethoxycarbonylamino)alkylenes comprising linear or branched C3-C20 alkylene groups, preferably C3-C9 alkylene group. Particular preference is also given to an alkylene substituted by methyl groups.

In addition, the further free-radically polymerizable monomer may be a reaction product of 3-hydroxypropyl methacrylate and trimethylhexamethylene diisocyanate, or a reaction product of 3-hydroxypropyl acrylate and trimethylhexamethylene diisocyanate.

Suitable further free-radically polymerizable monomers are available, for example, under the following trade or brand names: Ebecryl 230 (aliphatic urethane diacrylate), Actilane 9290, Craynor 9200 (diurethane acrylate oligomer), Ebecryl 210 (aromatic urethane diacrylate oligomers), Ebecryl 270 (aliphatic urethane diacrylate oligomer), Actilane 165, Actilane 250, Photomer 6210 (aliphatic urethane diacrylate), Photomer 6623 (hexafunctional aliphatic urethane resin), Photomer 6891 (aliphatic urethane triacrylate), UDMA, Roskydal LS 2258 (aliphatic urethane acrylate oligomer), Roskydal XP 2513 (unsaturated aliphatic urethane acrylate), Genomer 4256, Genomer 4267 (urethane acrylate), Genomer 4259 (aliphatic urethane dimethacrylate), RCX 18-059 (aliphatic urethane dimethacrylate), UN 1963CG (aliphatic urethane methacrylate), CN 1993CG (aliphatic urethane methacrylate), PRO 21252 (aliphatic urethane acrylate), H1391 (hydroxypropylurethane dimethacrylate), H1391 (urethane dimethacrylate), X851-1066 (urethane dimethacrylate IP-DI), X726-000 (PEG 400 extended urethane dimethacrylate), urethane methacrylate 11-70 and urethane methacrylate 14-774.

Particular preference is given to further free-radically polymerizable monomers having a structure of the following formula 32:

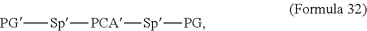

where
PG′=each independently selected from a free-radically polymerizable group, preferably selected from (NR³)OC—CR⁴═CH₂ and OOC—CR⁴═CH₂, more preferably selected from OOC—CR⁴═CH₂,
R³=selected from hydrogen, a C1-C8 alkyl group, an aryl group and an araliphatic group with C6-C8 carbon atoms, preferably selected from a C1-C8 alkyl group and a benzyl group;
R⁴=selected from hydrogen, C1-C4 alkyl group, preferably selected from hydrogen and a methyl group;
Sp′=each independently a spacer group selected from unbranched and branched alkylene having C1-C19 carbon atoms, which may additionally contain oxygen, sulfur and/or —OOC— in the carbon chain, preferably selected from methylene, *CH₂—(OC₂H₄)_{q}, *CH₂—(OC₃H₆)_{q}, where q=1-5 and the * symbol indicates the binding site to the PCA′,
   or Sp′ is absent;
PCA′=each independently a polycyclic group, preferably an aliphatic polycyclic group, preferably an aliphatic bi- or tricyclic group, more preferably a tricyclic group, most preferably tricyclo[5.2.1.0/2,6]decanylene.

The monomers of the formula 32 may in particular be bis(methacryloyloxymethyl)tricyclo[5.2.1.0/2,6]decane and bis(acryloyloxymethyl)tricyclo[5.2.1.0/2,6]decane.

Suitable further free-radically polymerizable monomers may also be selected from methyl (meth)acrylate, ethyl (meth)acrylate, 2-hydroxyethyl (meth)acrylate, butyl (meth)acrylate, benzyl (meth)acrylate, tetrahydrofurfuryl (meth)acrylate or isobornyl (meth)acrylate, p-cumylphenoxyethylene glycol methacrylate, bisphenol A di(meth)acrylate, bis-GMA, ethoxy or propoxylated bisphenol A dimethacrylate (e.g. SR-348c (Sartomer)) having three ethoxy groups, 2,2-bis[4-(2-methacryloyloxypropoxy)phenyl]propane, di-, tri- and tetraethylene glycol di(meth)acrylate, trimethylolpropane tri(meth)acrylate, pentaerythritol tetra(meth)acrylate, glycerol dimethacrylate and glycerol trimethacrylate, 1,4-butanediol di(meth)acrylate, 1,10-decanediol di(meth)acrylate and 1,12-dodecanediol di(meth)acrylate. Preference is given here to the monomers 1,6-hexanediol dimethacrylate, benzyl methacrylate, tetrahydrofurfuryl methacrylate or isobornyl methacrylate, p-cumylphenoxyethylene glycol methacrylate, 2,2-bis[4-(2-methacryloyloxypropoxy)phenyl]propane, bis-GMA and SR-348C (Sartomer).

Suitable further free-radically polymerizable monomers may be selected, for example, from N-monosubstituted and N-disubstituted acrylamides, such as N-ethylacrylamide and N,N-dimethacrylamide, bisacrylamides, such as N,N′-diethyl-1,3-bis(acrylamido)propane, 1,3-bis(methacrylamido)propane, 1,4-bis(acrylamido)butane, and 1,4-bis(acryloyl)piperazine.

The free-radically polymerizable compositions preferably comprise further free-radically polymerizable monomers or oligomers, the viscosity of which is below the viscosity of the free-radically polymerizable compound, represented by the structure of the formula 1. In such a case, the further free-radically polymerizable monomers or oligomers are called thinners. The thinners preferably have viscosities of less than 10 Pa·s, more preferably less than 5 Pa·s, more preferably less than 1 Pa·s. This is especially preferable and advantageous for the production of materials by means of tank-based photopolymerization.

Preferably, the free-radically polymerizable composition comprises one or more further free-radically polymerizable monomers or oligomers that are not covered by the at least one compound of the formula 1, in a proportion by mass of 0% to 99% by weight, preferably from 10% to 99% by weight, more preferably from 20% to 98% by weight, even more preferably from 35% to 90% by weight, based on the total mass of the polymerizable composition.

In a particular embodiment, it is preferable that the composition does not include any monomer or oligomer that has a bisphenol A structure. In particular, 2,2-bis[4-(2-hydroxy-3-(meth)acryloyloxypropoxy)phenyl]propane (bis-GMA) and/or ethoxylated bisphenol A di(meth)acrylate (bis-EMA) are absent.

For dental adhesives or else self-adhesive composite cements, it is possible in particular to use acidic monomers and/or water-soluble monomers. A typical proportion of acidic monomers is known from the prior art. Acidic monomers are compounds that have an acid group and a free-radically polymerizable group in one molecule. Examples of free-radically polymerizable unsaturated groups include: (meth)acryloyl, (meth)acrylamide, styryl, vinyl and allyl groups. Examples of acidic groups that may be represented in acidic monomers are: carboxylic acid, carboxylic anhydride, phosphate, thiophosphate, pyrophosphate, thiopyrophosphate, phosphonate, thiophosphonate and sulfonate groups. The acidic groups may also occur in the form of an acid chloride, an alkali metal salt, an alkaline earth metal salt or an ammonium salt. Suitable acidic monomers are, for example, 2-(methacryloyloxyethyl)phenyl hydrogenphosphate (phenyl-P); 2-hydroxyethylmethacryloyl dihydrogenphosphate (HEMA phosphate); dipentaerythritol pentamethacrylate phosphate (PENTA); di-2-hydroxyethylmethacryloyl hydrogenphosphate (di-HEMA phosphate); 10-methacryloyloxydecyl dihydrogenphosphate (MDP); 1,3-glycerol dimethacrylate phosphate (GDMAP), 2,5-dimethacryloyloxyethyloxycarbonyl-1,4-benzenedicarboxylic acid (PMDM), butane-1,2,3,4-tetracarboxylic acid di(2-hydroxyethylmethacryloyl) ester (TCB), 4-methacryloyloxyethyltrimellitic acid (4-MET), 4-methacryloyloxyethyltrimellitic anhydride (4-META), pyromellitic acid bis(glycerol dimethacrylate) (PMGDM) and 11-methacryloyloxy-1,1-undecanedicarboxylic acid (MAC-10).

In addition, further monomers used may also be free-radically polymerizable antibacterial monomers.

### c) Initiator or Initiator System

Suitable initiators or initiator systems are capable of initiating free-radical polymerization reactions. Such initiators and initiator systems are known to those skilled in the art.

Initiator systems consist of at least one initiator and at least one further compound, such as a co-initiator. These may be divided between different components of the polymerizable dental material. The dental material of the invention can be cured thermally, chemically, or photochemically, i.e. by irradiation with UV and/or visible light.

Suitable initiators may be, for example, photoinitiators. These are characterized in that they can bring about curing of the material by absorbing light in the wavelength range from 300 nm to 700 nm, preferably from 350 nm to 600 nm and more preferably from 380 nm to 500 nm, and optionally through additional reaction with one or more co-initiators. Preference is given here to using phosphine oxides, acylphosphine oxides, bisacylphosphine oxides and derivatives thereof, acylgermanes, acylsilanes and tin compounds, as described, for example, in EP 2649981 A1, 2017/055209 A1, WO 2017/060527 A1, EP 3068363 A1, US 2020/0087329 A1, EP 3868767 A1, EP 3293215 A1 and EP 3153150 A1, benzoin ethers, benzil ketals, acetophenones, benzophenones, thioxanthones, bisimidazoles, metallocenes, fluorones, α-dicarbonyl compounds, aryldiazonium salts, arylsulfonium salts, aryliodonium salts, ferrocenium salts, phenylphosphonium salts or a mixture of these compounds.

Particular preference is given to diphenyl(2,4,6-trimethylbenzoyl)phosphine oxide, phenylbis(2,4,6-trimethylbenzoyl)phosphine oxide, benzoin, benzoin alkyl ethers, benzil dialkyl ketals, α-hydroxyacetophenone, dialkoxyacetophenones, α-aminoacetophenones, isopropylthioxanthone, camphorquinone, phenylpropanedione, 5,7-diiodo-3-butoxy-6-fluorone, (η⁶-cumene) (η⁵-cyclopentadienyl)iron hexafluorophosphate, (η⁶-cumene) (η⁵-cyclopentadienyl)iron tetrafluoroborate, (η⁶-cumene) (η⁵-cyclopentadienyl)iron hexafluoroantimonate, substituted diaryliodonium salts, triarylsulfonium salts or a mixture of those compounds.

Co-initiators used for photochemical curing are preferably tertiary amines, borates, organic phosphites, diaryliodonium compounds, thioxanthones, xanthene, fluorenes, fluorones, α-dicarbonyl compounds, dicarbonyl systems as described in WO 2021/048313 A1, fused polyaromatics or a mixture of those compounds. Particular preference is given to N,N-dimethyl-p-toluidine, N,N-dialkylalkylaniline, N,N-dihydroxyethyl-p-toluidine, 2-ethylhexyl p-(dimethylamino)benzoate, ethyl p(dimethylamino)benzoate, butyrylcholine triphenylbutylborate or a mixture of those compounds.

Initiators used may also be what are called thermal initiators, which can bring about curing of the material by absorbing thermal energy at elevated temperatures. Preference is given here to using inorganic and/or organic peroxides, inorganic and/or organic hydroperoxides, diethyl α,α′-azobisisobutyrate, α,α′-azobis(isobutyronitrile), benzpinacols or a mixture of those compounds. Particular preference is given to diacyl peroxides such as benzoyl peroxide or lauroyl peroxide, cumene hydroperoxide, benzpinacol, 2,2′-dimethylbenzopinacol or a mixture of those compounds.

For chemical curing at room temperature, a redox initiator system is generally used that consists of one or more initiators and one or more co-initiators serving as activator. For storage stability reasons, individual components of an initiator system are incorporated in spatially separate components of the dental material of the invention, i.e. the material is a multicomponent, preferably two-component, material. Initiator(s) used are preferably inorganic and/or organic peroxides, inorganic and/or organic hydroperoxides, barbituric acid derivatives, malonylsulfamides, protic acids, Lewis or Bronsted acids or compounds that release such acids, carbenium ion donors, for example methyl triflate or triethyl perchlorate or a mixture of said compounds, and co-initiator(s) used are preferably tertiary amines, heavy metal compounds, especially compounds of the 8th and 9th group of the Periodic Table (“iron group and copper group”), compounds containing ionogenically bound halogens or pseudohalogens, for example quaternary ammonium halides, weak Bronsted acids, for example alcohols and water, or a mixture of those compounds.

The dental material of the invention may also include any conceivable combination of the initiators and co-initiators described above. An example of the above is what are known as dual-curing dental materials, which comprise both photoinitiators and optionally the corresponding co-initiators for photochemical curing and initiators and corresponding co-initiators for chemical curing at room temperature.

The polymerizable composition or the polymerizable dental material is preferably light-curing. In a preferred embodiment, the polymerizable composition comprises an initiator system which initiates the free-radical polymerization in the wavelength range of 395-700 nm, preferably in a wavelength range of 400-500 nm. A preferred initiator system contains camphorquinone (CQ) as initiator and tertiary aromatic and aliphatic amines as co-initiator. A preferred aliphatic amine is N,N-(dimethylamino)ethyl (meth)acrylate. Preferred aromatic co-initiators are aromatic amines, such as 2-ethylhexyl p-(dimethylamino)benzoate (EHA) or ethyl p-(dimethylamino)benzoate (EDAB). In addition to camphorquinone and a tertiary amine, the photoinitiator system may comprise a further synergist. Preferred synergists may be diaryliodonium salts as described in EP 3427716 A1, EP 3888616 A1, EP 3881818 A1 and M. Topa, J. Ortyl, Materials 13, 4093 (2020).

In another preferred embodiment, which is particularly suitable for 3D printing applications, the dental material of the invention contains an initiator system that initiates the free-radical polymerization in the wavelength range of 300-500 nm, preferably in a wavelength range of 350-420 nm, especially preferably 365-410 nm. Preferred initiator types are those that work by the Norrish type 1 mechanism.

The at least one initiator or the initiator system for the polymerization may be present in the polymerizable composition in a proportion by mass of 0% to 5% by weight, preferably from 0.01% to 5% by weight, based on the total mass of the polymerizable composition.

### d) Stabilizer

The free-radically polymerizable composition may contain one or more stabilizers. Such stabilizers are known to the skilled person.

Suitable stabilizers are preferably benzotriazoles, triazines, benzophenones, cyanoacrylates, salicylic acid derivatives, hindered amine light stabilizers (HALS) and mixtures thereof. The following are particularly suitable: o-hydroxyphenylbenzotriazoles, such as 2-2H-benzotriazol-2-yl)-4-methylphenol, 2-(5-chloro-2H-benzotriazol-2-yl)-4-methyl-6-tert-butylphenol, 2-(5-chloro-2H-benzotriazol-2-yl)-4,6-di-tert-butylphenol, 2-(2H-benzotriazol-2-yl)-4,6-di-tert-pentylphenol, 2-(2H-benzotriazol-2-yl)-4-methyl-6-dodecylphenol, 2-(2H-benzotriazol-2-yl)-4,6-bis(1-methyl-1-phenylethyl)phenol, 2-(2H-benzotriazol-2-yl)-6-(1-methyl-1-phenylethyl)-4-(1,1,3,3-tetramethylbutyl)phenol, 2-(2H-benzotriazol-2-yl)-4-(1,1,3,3-tetramethylbutyl)phenol and 3-(2H-benzotriazol-2-yl)-5-tert-butyl-4-hydroxybenzene propanoate, o-hydroxyphenyltriazines, such as 2-(2-hydroxy-4-hexyloxyphenyl)-4,6-diphenyl-1,3,5-triazine or 2-(2-hydroxy-4-[2-hydroxy-3-dodecyloxypropyloxy]phenyl)-4,6-bis(2,4-dimethylphenyl)-1,3,5-triazine, o-hydroxybenzophenones, such as 2-hydroxy-4-octyloxybenzophenone, cyanoacrylates, such as ethyl 2-cyano-3,3-diphenylacrylate, 2-ethylhexyl 2-cyano-3,3-diphenylacrylate and tetrakis[(2-cyano-3,3-diphenylacryloyl)oxymethyl]methane, hindered amine light stabilizers (HALS), such as N,N′-bisformyl-N,N′-bis-(2,2,6,6-tetramethyl-4-piperidinyl)hexamethylenediamine, bis(2,2,6,6-tetramethyl-4-piperidyl) sebacate, bis(1,2,2,6,6-pentamethyl-4-piperidyl) sebacate and methyl (1,2,2,6,6-pentamethyl-4-piperidyl)sebacate, salicylic esters and mixtures thereof.

Further suitable stabilizers are phenols such as hydroquinone monomethyl ether (HQME), 2,6-di-tert-butyl-4-methylphenol (BHT) or tert-butylhydroxyanisole (BHA). The stabilizer used may in particular be 2,6-di-tert-butyl-4-methylphenol (BHT).

The stabilizers may be present in the free-radically polymerizable composition in a proportion by mass of 0% to 5% by weight, preferably from 0.001% to 5% by weight, further preferably 0.005% to 2% by weight, based on the total mass of the polymerizable composition.

In one embodiment, the free-radically polymerizable composition may include stabilized free radicals. Suitable stabilized free radicals are preferably those such as 2,2,6,6-tetramethylpiperidinyloxyl (TEMPO) and bis(2,2,6,6-tetramethyl-4-piperidyl-1-oxyl) sebacate. Bis(2,2,6,6-tetramethyl-4-piperidyl-1-oxyl) sebacate is particularly preferred.

The stabilized free radicals are preferably present in the free-radically (photo)polymerizing compositions in a proportion by mass of 0.005% to 0.01% by weight, based on the total mass of the polymerizable composition.

### e) Fillers

The free-radically polymerizable composition may comprise fillers or filler particles. The filler particles are not defined as having a particular particle shape. Instead, fillers having a spherical, flake-like, platelet-like, needle-like, leaf-like or irregular shape may be of very good utility. The filler particles preferably have an average particle diameter of from 5 nm to 100 μm, more preferably from 5 nm to 50 μm.

Suitable fillers may be selected from a wide variety of materials commonly used in dental materials or products. Through the selection of the filler it is possible to adjust, for example, the fluidity, viscosity, consistency, color tone, radiopacity, and mechanical stability of a composition or dental material. The fillers can be broadly divided according to their chemical nature into three different classes: inorganic fillers, organic fillers, and organic-inorganic composite fillers. The fillers can be used not just individually, but also in combination with one other.

Inorganic fillers used may be ground powders of natural or synthetic glasses or crystalline inorganic substances in various sizes and states (monodisperse, polydisperse). Suitable materials include quartz, cristobalite, glass ceramics, feldspar, barium silicate glasses (for example those available under the Kimble Ray-Sorb T3000, Schott 8235, Schott GM27884, Schott G018-053, and Schott GM39923 trade names), barium fluorosilicate glasses, strontium silicate glasses, strontium borosilicate glasses (for example those available under the Ray-Sorb T4000, Schott G018-093, Schott G018-163, and Schott GM32087 trade names), lithium aluminosilicate glasses, barium glasses, calcium silicates, sodium aluminosilicates, fluoroaluminosilicate glasses (for example those available under the Schott G018-091 and Schott G018-117 trade names), zirconium or cesium boroaluminosilicate glasses (for example those available under the Schott G018-307, G018-308 and G018-310 trade names), zeolites, and apatites. The fillers preferably have a median particle size d50 of 0.01-15 μm, preferably a median particle size d50 of 0.2-5 μm, and more preferably a median particle size of 0.2-1.5 μm. It may be preferable that the median particle size d50 is between 0.1-0.5 μm. In such cases, it is particularly preferable that the median particle size d90 is less than 1.0 μm. In addition, discrete, non-agglomerated, non-aggregated, organically surface-modified nanoparticles may be used to achieve a more uniform filling of the dental material and to increase the hardness and abrasion resistance.

Nanoparticles in this context mean spherical particles having a median particle size of less than 200 nm. The median particle size is preferably less than 100 nm and more preferably less than 60 nm. The smaller the nanoparticles, the better they are able to fulfill their function of filling the cavities between the coarser particles. The materials for the nanoparticles are by preference oxides or mixed oxides and preferably selected from the group consisting of oxides and mixed oxides of the elements silicon, titanium, yttrium, strontium, barium, zirconium, hafnium, niobium, tantalum, tungsten, bismuth, molybdenum, tin, zinc, ytterbium, lanthanum, cerium, aluminum, and mixtures thereof. Preferred oxidic nanoparticles are not agglomerated. In order to permit good incorporation of the nanoparticles into the polymer matrix of a composite material, the surfaces of the nanoparticles are organically modified. The fillers are preferably surface treated using a silanizing agent. A particularly suitable adhesion promoter is methacryloyloxypropyltrimethoxysilane. Commercially available nanoscale, non-agglomerated and non-aggregated silica sols that can be used are marketed for example under the “Nalco Colloidal Silicas” (Nalco Chemical Co.), “Ludox colloidal silica” (Grace) or “Highlink OG” (Clariant) names.

Submicron fillers or microfillers consisting of agglomerated nanoscale particles may likewise be used, particularly if their specific surface area (determined by the Brunauer-Emmett-Teller method) is in the range between 100 to 400 m²/g. Fumed silica or wet-precipitated silica are preferred. Suitable non-surface-treated silicon dioxide fillers products that can be used are commercially available under the Aerosil™ (“OX50”, “90”, “130”, “150”, “200”, “300”, “380”, and “R8200” from Evonik Industries AG, Essen, Germany), Cab-O-Sil (“LM-150”, “M-5”, “H-5”, “EH-5” from Cabot Corp., Tuscola, IL), HDK™ (“S13”, “V15”, “N20”, “T30”, “T40”, Wacker-Chemie AG, Munich, Germany) and Orisil™ (“200”, “300”, “380”, Orisil, Lviv, Ukraine) names.

Particularly advantageous abrasion resistance and gloss resistance properties of the composition or a dental material can be achieved by using aggregated nanoscale particles based on mixed oxides of silicon dioxide and zirconium dioxide. A suitable filler can be produced by a process described for example in U.S. Pat. No. 6,730,156 (example A). The filler thus produced can then be surface treated by a method such as that described in U.S. Pat. No. 6,730,156 (for example production example B).

In order to achieve high filler contents in tandem with good esthetics and abrasion stability, it can be particularly advantageous to use spherical submicroparticles based on silicon-zirconium mixed oxides, such as those described in DE 19524362 A1 or US2020/0121564 A1.

The aggregated fillers preferably have a median particle size of 1-15 μm, preferably a median particle size of 1-10 μm, and more preferably a median particle size of 2-5 μm.

Appreciable amounts of selected radiopaque fillers may additionally be present. The addition of radiopaque particles to the polymerizable composition or dental material is advantageous, since this makes it possible to distinguish between healthy dental hard substance and the restoration. Suitable radiopaque fillers include particles of metal oxides, metal fluorides or barium sulfate. Oxides and fluorides of heavy metals of atomic number greater than 28 are preferred. The metal oxides and fluorides should be selected so as to affect the color of the restoration as little as possible. Metal oxides and metal fluorides of atomic number greater than 30 are more suitable. Suitable metal oxides are oxides of yttrium, strontium, barium, zirconium, hafnium, niobium, tantalum, tungsten, bismuth, molybdenum, tin, zinc, lanthanides (elements having an atomic number of 57 to 71), cerium, and combinations thereof. Suitable metal fluorides are for example yttrium trifluoride and ytterbium trifluoride. Suitable with particular preference here are irregularly shaped or spherical YbF3 or YF3 particles having an average primary particle grain size of from 40 nm to 1.5 μm and more preferably core-shell combination products having a YF3 or YbF3 core and SiO2 shell, very particularly preferably where the surface of the SiO2 shell is silanized. In particular, such a core-shell combination product has a refractive index of 1.48 to 1.54 and a measured median particle size of the agglomerated particles of between 0.5 and 5 μm.

Examples of suitable organic fillers are filled and unfilled, powdered polymers or copolymers based on polymethyl methacrylate (PMMA), polyethylene methacrylate, polypropylene methacrylate, polybutyl methacrylate, (PBMA), polyvinyl acetate (PVAc), polyvinyl alcohol (PVA), polyurethane (PU), polyurea, methyl methacrylate-ethyl methacrylate copolymer, ethylene-vinyl acetate copolymer, and styrene-butadiene copolymer. In addition, the organic filler may comprise a biologically active component, a specific pigment, a polymerization initiator, a stabilizer or similar that had been added during the production process. The organic fillers may be used alone or as mixtures.

Advantageous polishing properties in tandem with a higher filler level can be achieved in the composition or the dental materials when what are called organic-inorganic composite fillers are used. These fillers can be produced by processing a polymerizable monomer with an inorganic filler into a paste, then curing this by polymerization and then finely grinding prior to use as a filler. Preference is given here to using microfillers as inorganic filler. After grinding, the fillers preferably have a median particle size of 0.05-100 μm, more preferably a median particle size of 0.5-50 μm, and even more preferably a median particle size of 1-30 μm.

Median particle size is preferably determined by static and/or dynamic light scattering by the particles in a dispersion with a suitable dispersant (e.g. Coulter LS, Beckman Coulter GmbH, Krefeld, Germany and/or e.g. Zetasizer, Malvern Panalytical GmbIH, Kassel, Germany). Other methods employed with particular preference for this purpose are field-flow fractionation (e.g. AF2000 AT, Postnova Analytics GmbH, Landsberg, Germany) or a calibration with particle size standards. Median particle size can also be determined by microscopy, especially electron microscopy. The figure reported is preferably the absolute particle size and preferably the d50 value.

It is preferable that the fillers are surface-modified in the free-radically polymerizable composition or in the dental material. This is done for example by subjecting the described inorganic or organic-inorganic composite fillers, prior to use, to a surface treatment in order to improve the compatibility, affinity, and the incorporation of the fillers into the resin mixture. This treatment organically modifies the surfaces of the inorganic particles, i.e. the surfaces have organic structural elements. All methods known to those skilled in the art can be employed here. Silanizing agents are preferred for inorganic fillers bearing surface OH groups. Examples here include γ-methacryloyloxyalkyltrimethoxysilanes (number of carbon atoms between the methacryloyloxy group and the silicon atom: 3 to 12), γ-methacryloyloxyalkyltriethoxysilanes (number of carbon atoms between the methacryloyloxy group and the silicon atom: 3 to 12) or silicone compounds such as vinyltrimethoxysilane, vinylethoxysilane, and vinyltriacetoxysilane. The silanizing agent is particularly preferably methacryloyloxypropyltrimethoxysilane.

Inorganic fillers that have few surface OH groups or none at all are preferably used with different surface modifiers, for example surface-treated with titanates, aluminates, zircoaluminates, surfactants, fatty acids, organic acids, inorganic acids or metal alkoxides. Surface modification agents for salts of barium, strontium, and rare earth metals are particularly preferably organic compounds bearing N-, P-, S- and/or O-containing functional groups (for example polyols, sulfoxides, phosphinic esters, phosphonic esters, trialkylphosphines, carboxylic acids and carboxylic esters). Particularly suitable here is 10-methacryloyloxydecyl dihydrogenphosphate.

Particularly in the case of agglomerated silicon-dioxide-based nanofillers, the surface modifications may consist of groups reactive to free radicals, such as the abovementioned methacryloyloxyalkyl groups, or of groups unreactive to free radicals. Suitable unreactive groups are for example trimethylsilyl, dimethylsilylene or methylsilylidene groups, which can be applied to the surface by silanization with hexamethyldisilazane, dimethyldimethoxysilane or methyltrimethoxysilane, for example. Suitable non-reactive surface-modified agglomerated nanofillers are commercially available under the Aerosil R8200, Aerosil R812S, Aerosil R805, Aerosil R202, and Aerosil R974 (Evonik Industries AG, Essen, Germany) or HDKH2000 and HDKH200/4 (Wacker Chemie, Burghausen, Germany) names. More preferably, the agglomerated nanofillers may be modified with groups that are reactive in free-radical processes, for example methacryloyl groups. A commercial agglomerated nanofiller product modified to be reactive to free radicals is available under the Aerosil R7200 name (Evonik Industries AG, Essen, Germany).

The agglomerated nanofillers may preferably be present in largely deagglomerated form, as described for example in EP 1720206.

The free-radically polymerizable composition of the invention may contain a proportion by mass of filler or filler particles of 0% to 95% by weight, preferably from 1% to 95% by weight, more preferably from 5% to 92% by weight, even more preferably from 15% to 85% by weight, based on the total mass of the polymerizable composition.

The amount of the filler fraction may be chosen in accordance with the indication for the dental product. For instance, maximum amounts of fillers may be used for firm, modelable filling composites, for dental compositions for producing inlays, onlays or overlays, and for compositions for producing dental CAD-CAM materials. These compositions generally have filler contents of from 75% by weight to 92% by weight based on the overall composition. Free-flowing dental composites, luting composites, core build-up materials, crown materials and bridge materials generally have an average filler content ranging from 40% to 80% by weight, based on the overall composition, while dental lacquers, dental sealing materials, dental infiltrants or dental adhesives employ fillers in a content ranging from 1% to 40% by weight, based on the overall composition. The filler content ranges stated above should be understood as guide values only; departures from this are also possible, depending on the selected filler.

The free-radically polymerizable composition, in a preferred embodiment, contains a selection of the microfillers such that there is a minimum difference in refractive index between the microfillers and the remaining components of the polymerized composition (polymer matrix). Preferably, Δn≤0.03, more preferably ≤0.02, more preferably ≤0.01.

### f) Customary Dental Additives

The free-radically polymerizable composition may contain further dental additives. Suitable customary dental additives are preferably those which may be present in free-radically polymerizable dental and orthodontic materials. Customary dental additives are known to those skilled in the art.

Customary dental additives used may include, for example, solvents or solvent mixtures. For instance, preference is given to the use of a mixture of water and water-miscible solvents, such as ethanol or acetone, for the production of dental adhesives.

In addition, further constituents of the free-radically polymerizable composition may, for example, be pharmacologically active compounds such as antibacterial compounds, chlorhexidine or other enzyme-inhibiting active substances.

Customary dental additives present in the composition may also include one or more fluoride-releasing substances in finely divided particulate form. Fluoride-releasing substances may be water-soluble fluorides such as sodium fluoride or amine fluoride. Other suitable fluoride-releasing substances are sparingly soluble fluorides of main group 2. Fluoride-containing glasses are suitable sources of fluoride too.

Other suitable additives are finely particulate substances that release calcium and/or phosphate and accordingly have a remineralizing effect. Suitable remineralizing substances are calcium-phosphate compounds such as hydroxyapatite, brushite, monocalcium phosphate, fluoroapatite, and bioactive glasses such as those mentioned in DE10111449A1, DE102005053954A1 or U.S. Pat. No. 9,517,186B2.

The dental material of the invention may comprise a colorant or colorant mixture selected from fluorescent dyes, fluorescent pigments, organic color pigments, inorganic color pigments, and mixtures thereof.

A fluorescent colorant or pigment is preferably an organic fluorescent dye or organic fluorescent pigment, in particular a non-polymerizable organic fluorescent colorant optionally comprising esters of aryl carboxylic acids, such as diethyl 2,5-dihydroxyterephthalate, aryl carboxylic acids, coumarin, rhodamine, naphthalimide or derivatives thereof. Examples of inorganic fluorescent pigments include CaAl₄O₇:Mn²⁺ (Ba0.98Eu0.02)MgAl₁₀O₁₇, BaMgF₄:Eu²⁺, and Y(1.995)Ce(0.005)SiO₅. Color pigments included in the dental material of the invention may include organic pigments and also inorganic pigments, such as N,N′-bis(3,5-xylyl)perylene-3,4:9,10-bis(dicarboximide), copper phthalocyanine, and titanate pigments, in particular chromium antimony titanates (rutile structure), spinel black, in particular pigments based on iron oxide (Fe₂O₃) or iron oxide black (Fe₃O₄) in which iron is partly replaced by chromium and copper or nickel and chromium or manganese, zinc iron chromite brown spinel ((Zn,Fe)(Fe,Cr)₂O₄) cobalt zinc aluminate blue spinel and/or titanium oxide.

The customary dental additives may be present in the polymerizable composition in a proportion by mass of 0% to 5% by weight, preferably from 0.001 to 5% by weight, based on the total mass of the free-radically polymerizable composition.

The invention has the advantage that the free-radically polymerizable compound of the invention and also the free-radically polymerizable composition of the invention overcome the above-detailed disadvantages of the prior art. The free-radically polymerizable compounds and the free-radically polymerizable composition have excellent properties that are necessary or desirable in particular for the production of dental materials.

In addition, the invention has the surprising advantage that the free-radically polymerizable compounds of the invention can be synthesized by the production process of the invention not only inexpensively but also in high purity and without unwanted discoloration. Furthermore, the free-radically polymerizable compositions have a high conversion of the monomers in the free-radical polymerization. As a result, dental materials and devices produced from the free-radically polymerizable compositions contain only a small residual monomer content.

The free-radically polymerizable composition may additionally be produced from monomers that are readily obtainable and have a reduced toxicity potential. Since the free-radically polymerizable compositions preferably do not include any compounds containing a bisphenol A group and/or another bisphenol group, it can also be very substantially ruled out that the dental materials obtained and devices produced therefrom will be harmful to health.

The use of the free-radically polymerizable compounds or of the free-radically polymerizable composition for production of a dental material leads to reduced polymerization shrinkage in tandem with good mechanical properties in the dental material obtained. This means that the free-radically polymerizable compositions have low polymerization shrinkage and reduced shrinkage stress. The free-radically polymerized compositions and the dental materials produced therefrom have good mechanical properties, such as, in particular, very good flexural strength and tensile strength, and good fracture resistance. These advantageous properties of the free-radically polymerizable compound of the invention and the corresponding compositions are surprising in view of the molecular sizes and structures of the free-radically polymerizable compounds, since the skilled person would expect an associated reduction in crosslinking density and flexural strength.

The free-radically polymerizable compounds or compositions surprisingly have a refractive index particularly suitable for production of dental materials containing fillers that are customary in the dental sector and having the desired optical properties, in particular with regard to translucence or opacity.

In addition, the free-radically polymerized compositions (i.e. the dental material produced) have only low water solubility and low water absorption, which is likewise advantageous.

The invention likewise provides for the use of the free-radically polymerizable compound, preferably as claimed in any of claims 1 to 7, or of the free-radically polymerizable composition, preferably as claimed in any of claims 9 to 12, for production of a polymerizable dental material, preferably a dental composite, dental cement, self-adhesive dental cement, dental lacquer, core build-up material, root canal filling material, filling material, lining material, luting material, crown material, bridge material, restoration material, orthodontic material and/or prosthesis material.

In this case, the filling material may be a modelable and/or free-flowing filling material, preferably a modelable filling material.

In a preferred embodiment, the polymerizable dental material is used as a 3D printing material. In this way, the polymerizable dental material can be used to produce, with a 3D printer, orthodontic materials, aligners, rail materials, prosthesis base materials, modeling materials, crown and bridge materials, drilling templates, gingiva masks, spoon materials, mouth guards and/or veneers.

Furthermore, the present invention also relates to a dental material produced from a free-radically polymerizable composition of the invention, preferably as claimed in any of claims 9 to 12.

The invention further provides a free-radically polymerizable dental material of the invention for use in a therapeutic method as dental composite, dental cement, dental lacquer, filling material, lining material, luting material, core build-up material, root canal filling material, crown material, bridge material, restoration material, orthodontic material and/or prosthesis material.

The invention likewise provides a cured dental material produced from a polymerizable composition of the invention, preferably as claimed in any of claims 9 to 12. The cured dental material can be produced in a process in which a free-radically polymerizable composition is provided, which is completely or partly hardened or cured. In a particular embodiment, the free-radically polymerizable composition can be 3D-printed prior to curing.

The invention is now described by way of example by a few advantageous embodiments with reference to the appended figure.

The figures show:

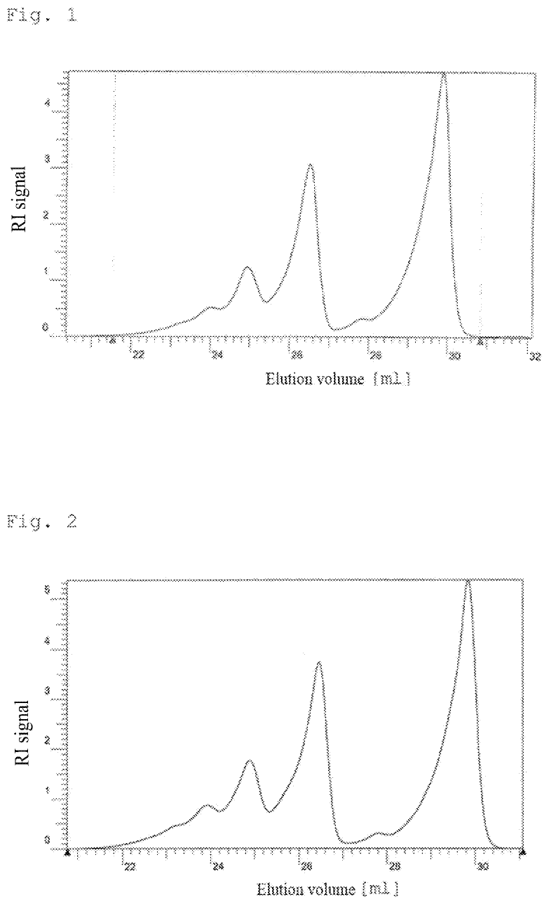

FIG. 1: GPC diagram showing the peak of an inventive free-radically polymerizable compound of the formula 31.

FIG. 2: GPC diagram showing the peak of an inventive free-radically polymerizable compound of the formula 33

### EXAMPLES

### 1. Chemicals and Pretreatment/Use Thereof

The following chemicals were used in the production of the examples (table 1) and processed as described in this section before further use.

TABLE 1
Compounds and substances used in the examples.

| | |
|---|---|
| TCD-DM | 4,8-bis(hydroxymethyl)tricyclo[5.2.1.0^{2,6}]decane, |
| | isomer mixture 96%, CAS 26896-48-0, Sigma Aldrich |
| TCDDA | Genomer 1231 (tricyclodecanedimethanol diacrylate), |
| | CAS 42594-17-2, Rahn AG, Zurich, Switzerland |
| TCD-MA | (hydroxymethyl)(acryloyloxymethyl)tricyclo |
| | [5.2.1.0^{2,6}]decane |
| IPDI | isophorone diisocyanate, >99%, CAS 4098-71-9; TCI |
| | Deutschland GmbH, Eschborn, Germany |
| H12MDI | 4,4′-diisocyanatodicyclohexylmethane, >90%, |
| | CAS: 5124-30-1; TCI Deutschland GmbH |
| TMXDI | 1,3-bis(1-isocyanato-1-methylethylbenzene), >97%, |
| | CAS: 2778-42-9; TCI Deutschland GmbH |
| H6XDI | 1,3-bis(isocyanatomethyl)cyclohexane, >99%, |
| | CAS: 38661-72-2; Sigma Aldrich |
| TMDI | trimethylhexamethylene diisocyanate, 97%, CAS: |
| | 28679-16-5, abcr GmbH, Karlsruhe, Germany |
| CALB | Candida Antarctica Lipase B, immobilized on |
| | Immobead 150; Sigma Aldrich |
| MTBE | methyl tert-butyl ether, 99%, CAS: 1634-04-4 |
| THF | tetrahydrofuran anhydrous, min. 99.9% |
| Toluene | toluene for analysis, 96%, CAS 108-88-3 |
| Toluene-t | toluene dry, 99.8%, CAS 108-88-3 |
| | cyclohexane, >99%, CAS 110-82-7 |
| | ethyl acetate, >99.8%, CAS 141-78-6 |
| BHT | 2,6-di-tert-butyl-4-methylphenol for synthesis, >99%, |
| | CAS 128-37-0 |
| DMTND | dimethyltin dineodecanoate, CAS: 68928-76-7 |
| TCDDMA | bis(methacryloyloxymethyl)tricyclo[5.2.1.0^{2,6}]decane; |
| | CAS 43048-08-4 |
| UDMA | 7,7,9-(or 7,9,9-)trimethyl-4,13-dioxo-3,14-dioxa- |
| | 5,12-diazahexadecane-1,16-diol dimethacrylate, CAS |
| | 72869-86-4 |
| Bis-GMA | propane-2,2-diylbis[4,1-phenylenoxy(2-hydroxypropane- |
| | 3,1-diyl)] bis(2-methylprop-2-enoate), CAS |
| | 1565-94-2 |
| TEDMA | triethylene glycol dimethacrylate, CAS 109-16-0 |
| CQ | camphorquinone |
| EHA | 2-ethylhexyl p-(dimethylamino)benzoate |
| BaF | G018-053 dental glass (median particle size |
| | 0.7 μm, 6% by weight of silane); Schott AG, Mainz, Germany |

### Drying of TCD-DM

The TCD-DM was dried by azeotropic distillation with about 2.5 times the volume of toluene until a solution of 83.6% by weight of TCD-DM in toluene was obtained. The TCD-DM was used in the form of this solution, unless stated otherwise, with weight figures relating to the TCD-DM.

### 2. Methods

### Thin-Layer Chromatography (TLC)

TLC was conducted with silica as stationary phase (TLC plates: Polygram Sil G/UV₂₅₄, Macherey-Nagel GmbH & Co. KG, Germany). A sample of the substance to be analyzed was diluted 1:10 with tetrahydrofuran. The eluent used was a mixture of cyclohexane and ethyl acetate 2:1. The developed TLC plate was viewed under UV light (wavelength 254 nm) and then stained in an iodine chamber.

### Drying Loss

Drying loss was determined gravimetrically. For this purpose, a sample was dried at 110° C. for 2 hours or at 50° C. for 2 hours in a convection oven and the residue was weighed.

### Viscosity

Viscosity measurements were performed with a rotary viscometer (Kinexus type, Malvern Instruments GmbH, Germany) with a plate-plate geometry (d=25 mm, gap h=0.1 mm, shear stress ramp 1-50 Pa) at 23° C. and the viscosity was determined at a shear stress of 50 Pa.

### FT-IR Spectroscopy

FT-IR spectra (iS10 and iS20 models, Thermo Scientific Nicolet) were produced with one ATR unit each. 32 scans were acquired with a resolution of 4 cm⁻¹.

### Liquid Chromatography-Mass Spectrometry Coupling (HPLC-MS)

This analysis was performed on a Waters Alliance 2695 HPLC system. The following column was used for separation: 250/2 Nucleodur C8 ec. The column temperature was 20° C. Elution was effected at a flow rate of 0.2 ml/min with a gradient of the following eluents: 0.1% formic acid in acetonitrile (A) and 0.1% formic acid in water (B).

| Time [min] | 0 | 0.5 | 40 | 45 | 50 | 60 |
|---|---|---|---|---|---|---|
| Proportion of | 70 | 70 | 10 | 10 | 70 | 70 |
| B [%] | | | | | | |

Detection was effected with a Waters Micromass ZQ mass detector. Ionization was effected in ESI+ mode.

### HPLC Method for Quantification of TCDDA

TCDDA content_{HPLC} in % by weight in the samples was determined on a Thermo Ultimate 3000 HPLC system. The following column was used for separation: 250/4 LiChrospher RP Select B (5 μm). The column temperature was 20° C. Elution was effected at a flow rate of 1 ml/min with a gradient of the following eluents: water (A) and methanol (B).

| Time [min] | 0 | 0.5 | 14.5 | 15.0 | 20.0 |
|---|---|---|---|---|---|
| Proportion of B | 70 | 70 | 95 | 70 | 70 |
| [%] | | | | | |

Detection was effected with a UV detector at 205 nm. The TCDDA reference sample concentration was about 25 mg/50 ml of methanol. The sample concentration of the examples was about 15 mg/10 ml of methanol. 3 μl of sample was injected for the measurement.

The content of monomers and oligomers of the invention is calculated in the samples as follows:

$$Content of monomer + oligomer of the invention ( \% by wt . ) = 100 \% - dry loss ( \% by wt . ) - TCDDA {content}_{HPLC} ( \% by wt . )$$

### Gel Permeation Chromatography (GPC)

The GPC was conducted with a GPC Agilent 1200 with RI detector (from PSS, Germany). The following column combination (from PSS, Germany) was used: precolumn/100 Å/100 Å/1000 Å. The eluent was THF with a flow rate of 1 ml/min. The sample concentration was about 5 mg/ml. The GPC was conducted at 20° C.

### Determination of Refractive Indices

The refractive indices of uncured compositions were determined by Abbe refractometer AR (A. Kruss Optronik, Germany) at 23° C.±1° C. versus air based on the D line of sodium light. A triple determination was conducted in each case, and the average was calculated.

The refractive index of cured compositions was determined according to ISO 489:1999 by Abbe refractometer AR (A. Kruss Optronik) on test specimens. A drop of cinnamon oil was applied to the specimen and brought into contact with the measuring prism of the refractometer. For each specimen, a triple determination was conducted and the average was calculated. For production of the specimens, the composition was placed in a steel mold (8 mm×20 mm×0.5 mm), which was positioned on a slide covered with a clear, colorless polyester film (Hostaphan®). Another film, followed by another slide, was placed onto the resin in a bubblefree manner, fastened with clamps and exposed by means of a light polymerization device (Hi-Lite Power®; Heraeus Kulzer) for 90 s. The cured specimen was then removed from the mold.

### Determination of Flexural Strength (FS) and Modulus of Elasticity (ME)

For the determination of flexural strength and modulus of elasticity, test specimens were produced analogously to ISO 4049:2009. In a departure thereof, the test specimens were produced by exposure with a light polymerization device (Hi-Lite Power®; Heraeus Kulzer). For this, the dental composites in the test specimen shapes (40 mm×2 mm×2 mm) were each irradiated from both sides for 90 s. The test specimens were stored in distilled water at 37° C. for 24 hours. Flexural strength and modulus of elasticity were determined using a Zwick universal tester (model Z010 or Z2.5, Zwick-Roell, Germany). The figures reported are the mean and standard deviation.

### Measurement of Volume Shrinkage of Resin Mixtures (Liquid Pycnometer)

The volume shrinkage of resin mixtures was determined with liquid pycnometers (Blaubrand, Brand GmbH+Co KG, Germany) via the change in density before and after curing at 20° C.

First, the density of the uncured compositions was determined with a 10 ml liquid pycnometer. Higher-viscosity compositions (viscosity >10 Pas) were equilibrated beforehand to 60° C. in a heating cabinet, in order to be able to subsequently introduce them into the pycnometer with a minimum level of air bubbles. Air bubbles were additionally removed by the applying of a reduced pressure in the desiccator. The filled pycnometer was then equilibrated to 20° C. Once the measurement temperature had been attained, the mass of the filled pycnometer was measured and the density of the uncured composition was determined therefrom.

For the determination of the density of the cured compositions, cuboidal specimens (35 mm×20 mm×3 mm) were produced. For this purpose, the uncured compositions were introduced without air bubbles into corresponding specimen molds and cured from above and beneath with the light polymerization device (Hi-Lite Power®; Heraeus Kulzer) for 90 s. The cured specimens were stored at 23° C. for 24±2 hours.

For the determination of density of the cured compositions, the mass of the hardened test specimen m1 and the empty weight of the pycnometer m0 were determined. Then the pycnometer was filled to an extent of 4/5 with distilled degassed water and equilibrated to 20° C., before the cured specimen was immersed in the pycnometer. The pycnometer was then completely filled up with water. On attainment of the measurement temperature of 20° C., the pycnometer was closed and dried externally, the mass m3 of the pycnometer filled with water and test specimen was measured, and the density of the cured test specimen ρ_{NA} was determined therefrom according to equation 1:

$${ρ}_{NA} = \frac{m 1 \times 0.9982}{m 1 + 0.9982 \times V 2 + m 0 - m 3} \qquad ( equation 1 )$$

V2=specified volume of the pycnometer used at 20° C.

In this way, 3 hardened test specimens in each case were analyzed.

Volume shrinkage (VS) as the average (MW_{VS}) was calculated from the difference in density before (ρVA) and the average of the densities after curing (MWρNA) (equation 2).

$${MW}_{VS} = 100 \% \times \frac{MW ρ NA - ρ VA}{MW ρ NA} \qquad ( equation 2 )$$

The standard deviation of the volume shrinkage SD_{VS} was determined according to equation 3.

$${SD}_{VS} = 100 \times {ρ}_{VA} \times SD {ρ}_{NA} / {MW}^{2} {ρ}_{NA} \qquad ( equation 3 )$$

### Measurement of the Volume Shrinkage of Composites (Gas Pycnometer)

The volume shrinkage of the composites was determined with a helium gas pycnometer (AccuPyc III 1340, Micromeritics, USA) via the change in the density before and after curing.

3 determinations of density of the uncured composite were conducted. For each measurement, about 0.3 to 0.4 g of the composite was placed in the measuring chamber of the gas pycnometer in a cavity-free manner. The weight of the uncured composite was determined using a balance and the density of the uncured composite was determined by the measurement of volume by the gas pycnometer using the AccuPYKII 1340 software.

Subsequently, 3 determinations of density of the cured composite were conducted. For a determination of density, 2 cylindrical specimens (h=2 mm, D=8 mm) of the composite were produced by curing (90 s from each side) with the HiLite Power light polymerization device. The specimen molds were filled without air bubbles and covered with a slide above and below during curing. After curing, the specimens were demolded, deburred, cleaned with ethanol and dried with compressed air. Subsequently, the two test specimens were placed one above another in the measuring chamber of the gas pycnometer. The weight of the cured composite was determined using a balance and the density of the cured composite was determined from the measurement of volume by the gas pycnometer using the AccuPYKII 1340 software. Density was determined within 15-60 minutes after curing.

Volume shrinkage (VS) as the average (MW_{VS}) was calculated from the difference in the averages of the density of the composite before curing (MWρVA) and the average of the densities after curing (MWρNA) (equation 4).

$$Average volume shrinkage {MW}_{VS} = 100 \% \times \frac{MW ρ NA - ρ VA}{MW ρ NA} \qquad ( equation 4 )$$

The standard deviation of volume shrinkage SD_{VS} was ascertained by equation 5 from the standard deviation SDρ_{NA} of the density of the cured composite and the standard deviation SDρ_{VA} of the density of the uncured composite, and the corresponding averages MWρNA and MWρVA.

$${SD}_{VS} = 100 / MW {ρ}_{NA}^{2} \times √ ( ( MW {ρ}_{NA}^{2} \times SD {ρ}_{VA}^{2} ) + ( MW {ρ}_{VA}^{2} \times SD {ρ}_{NA}^{2} ) ) \qquad ( equation 5 )$$

### Measurement of Conversion

Conversion was determined by FT-NIR spectroscopy (Nicolet iS20, Thermo Scientific) before and after polymerization of a composition in transmission geometry. The conversion or progression of the polymerization reaction was determined by measuring the decrease in size of the C═C overtone band at 6160-6170 cm⁻¹.

For this purpose, the unhardened, i.e. unpolymerized, composition or the composite was brought into a cylindrical specimen shape (D=15 mm, h=1.0 mm), covered with slides above and beneath, and then attached to an IR holder. A spectrum of 12 scans was acquired. The unhardened, i.e. unpolymerized, composition or composite was then cured from each side with the light polymerization device (Hi-Lite Power®; Heraeus Kulzer) for 90 s. The FT-NIR spectrum of the polymerized composition was then recorded within not more than 60 minutes. The integrals of the C═C overtone band at 6160-6170 cm⁻¹ were then determined from the two spectra using the IR software Omnic (Series 9.11.727; Thermo Scientific). Conversion C in % is calculated from the two integrals by equation 6:

$$Conversion in \% = 100 \% - \frac{Peak area Polymerized material}{Peak area Unpolymerized material} \times 100 \% \qquad ( equation 6 )$$

### 3. Synthesis Examples

### Example 1

### Enzymatic Transesterification of TCDDA and TCD-DM to TCD-MA

A 250 ml round-neck flask with magnetic stirrer bar was charged with a solution of 24.00 g of TCD-DM and 92.16 g of TCDDA in 80 ml of MTBE. 2 g of CALB was added and the flask was closed with a CaCl₂ drying tube. The preparation was stirred gently in a water bath at 40° C. for 42 hours and left to stand at room temperature (RT) for 128 hours. The preparation was filtered and washed twice with little MTBE, and the filtrate was concentrated on a rotary evaporator and then dried under a fine vacuum. What remained was a colorless oil of low viscosity. Yield: 104.5 g

TCD-DM (9.55), TCD-MA (21.5) and TCDDA (33.6) were detected by HPLC-MS. The respective retention time in minutes is given in brackets. The drying loss (2 hours, 50° C.) was 0.34% by weight.

### Example 2

### Synthesis of Free-Radically Polymerizable Compounds of a Structure of the Formula 33

To an initial charge of 50 g of oil according to example 1 in 50 g of THF in a 250 ml two-neck flask with magnetic stirrer bar, internal thermometer and dropping funnel with a CaCl₂ drying tube was slowly added dropwise, at RT, 9.55 ml of IPDI. Subsequently, 10 μl of catalyst solution (DMTND in toluene-t, weight ratio 1:1) was added and the mixture was stirred at 40° C. for 8 hours and at RT for 85 hours. For conversion of remaining isocyanate groups, 5.77 g of oil from example 1 in 5.77 g of THF was added and the mixture was stirred at 40° C. for 2 hours. Subsequently, isocyanate groups were no longer detectable by FT-IR spectroscopy.

The reaction mixture was transferred to a 250 ml one-neck flask, and THF was very substantially removed on a rotary evaporator at 25 mbar and water bath temperature 50° C. Further volatile constituents were removed under fine vacuum at 50° C. What remained was a clear, colorless oil.

Yield: 57.68 g

Drying loss (2 hours, 110° C.): 1.69%

By HPLC-MS, TCDDA (M+H⁺=305) was detectable at a retention time of 34.0 min, and a compound of the formula 33 (M+H⁺=724) at a retention time of 45.4 min.

The elution profile of the GPC performed is shown in FIG. 1. TCDDA had an elution volume of 29.8 ml, the compounds of structure 33 with x=1 an elution volume of 26.3 ml, and those with x=2-3 elution volumes of 25.0 and 24.0 ml respectively.

TCDDA content_{HPLC}: 48.09% by wt.

Content of compounds of formula 31: 50.22% by wt.

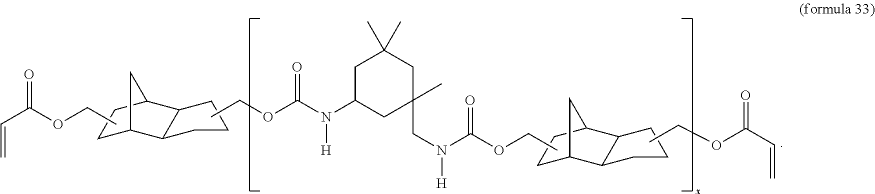

### Example 3

### Synthesis of Free-Radically Polymerizable Compounds of a Structure of the Formula 34

To an initial charge of 30 g of oil from example 1 in 30 g of THF in a 250 ml two-neck flask with magnetic stirrer bar, internal thermometer and dropping funnel with a CaCl₂ drying tube was slowly added dropwise, at RT, 7.17 g of H12MDI. Subsequently, 10 μl of a catalyst solution (DMTND in toluene-t, weight ratio 1:1) was added and the mixture was stirred at 40° C. for 4 hours and at RT for 19 hours. For conversion of remaining isocyanate groups, a further 11.61 g of oil from example 1 in 11.61 g of THF was added stepwise (4 steps). After each addition, the mixture was stirred at 45° C. for 3 hours and at RT for 18 hours. Subsequently, isocyanate groups were no longer detectable by FT-IR spectroscopy.

The reaction mixture was transferred to a 250 ml one-neck flask, and THF was very substantially removed on a rotary evaporator at 25 mbar and water bath temperature 50° C. Further volatile constituents were removed under fine vacuum at 50° C. What remained was a clear, colorless oil.

Yield: 42.79 g

Drying loss (2 hours, 110° C.): 2.07%

By HPLC-MS, TCDDA (M+H⁺=305) was detectable at a retention time of 33.6 min, and a compound of the formula 34 (M+H⁺=764) at a retention time of 48.6 min.

GPC: TCDDA had an elution volume of 29.6 ml, the compounds of structure 34 with y=1 an elution volume of 26.0 ml, and those with y=2 and 3 elution volumes of 24.3 and 23.2 ml respectively.

TCDDA content_{HPLC}: 47.35% by wt.

Content of compounds of the formula 32: 50.58% by wt.

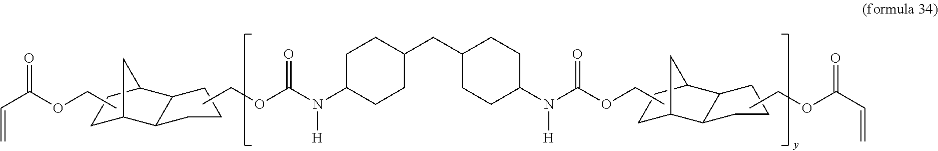

### Example 4

### Synthesis of Free-Radically Polymerizable Compounds of the Formula 35

A 250 ml three-neck flask with magnetic stirrer bar, internal thermometer, reflux cooler, dropping funnel and CaCl₂ drying tube was initially charged with 11.35 g TMDI in 25 ml of THF and 50 μl of catalyst solution (DMTND in toluene-t, 50:50 w/w). A solution of 50.0 g of oil from example 1 in 25 ml of THF was slowly added dropwise at RT while stirring. The mixture was then stirred at 45° C. for 5 hours and at RT for a further 18 hours.

For conversion of remaining isocyanate groups, a further 6.44 g of oil was added stepwise (4 steps) until isocyanate groups were no longer detectable by FT-IR spectroscopy. After each addition, the mixture was stirred at 45° C. for 5 hours and at RT for 18 hours.

The reaction mixture was transferred to a 250 ml one-neck flask and 0.01 g BHT was added. The majority of THF was removed on a rotary evaporator. Solvents and other volatile constituents were further removed under fine vacuum at 50° C. What remained was a clear, colorless oil. Yield: 55.94 g

Drying loss (2 hours, 110° C.): 0.99%

By HPLC-MS, TCDDA (M+H⁺=305) was detectable at a retention time of 33.7 min, and a compound of the formula 35 (M+H⁺=712) at a retention time of 45.2 min.

The elution profile of the GPC performed is shown in FIG. 2. TCDDA had an elution volume of 29.8 ml, the compound of the structure of the formula 35 with z=1 an elution volume of 26.5 ml, and higher oligomers (z=2-4) elution volumes of 24.9, 23.9 and 23.2 ml respectively.

TCDDA content_{HPLC}: 44.97% by wt.

Content of compounds of formula 35: 54.04% by wt.

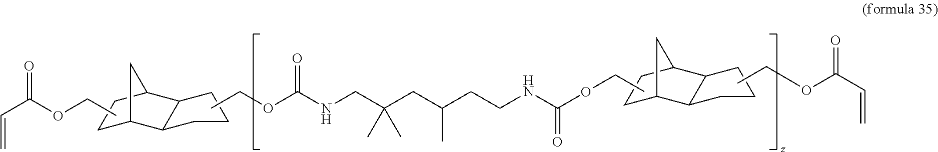

### Example 5

### Synthesis of Free-Radically Polymerizable Compounds of the Structure of the Formula 36

To an initial charge of 30 g of oil from example 1 in 30 g of THF and 0.005 g of BHT in a 250 ml two-neck flask with magnetic stirrer bar, internal thermometer and dropping funnel with a CaCl₂ drying tube was slowly added dropwise, at RT, 6.67 g of TMXDI. 10 μl of a catalyst solution (DMTND in toluene-t, weight ratio 1:1) was added and the mixture was stirred at 40° C. for 4 hours and at RT for 18 hours. For conversion of remaining isocyanate groups, a further 5.22 g of oil from example 1 and 10 μl of the catalyst solution were added and the mixture was stirred at 40° C. for 5.5 hours and at RT for 18 h. Subsequently, isocyanate groups were no longer detectable by FT-IR spectroscopy.

The reaction mixture was transferred to a 250 ml one-neck flask, and THF was very substantially removed on a rotary evaporator at 25 mbar and water bath temperature 50° C. Further volatile constituents were removed under fine vacuum at 45° C. What remained was a clear, colorless oil.

Yield: 33.75 g

Drying loss (2 h, 110° C.): 1.49%

By HPLC-MS, TCDDA (M+H⁺=305) was detectable at a retention time of 34.5 min, and a compound of the structure of the formula 36 (M+H⁺=746) at a retention time of 45.4 min.

GPC: TCDDA had an elution volume of 29.7 ml, the compound of the structure of the formula 36 with 1=1 an elution volume of 26.5 ml, and that with 1=2-4 elution volumes of 24.9, 24.0 and 23.2 ml respectively.

TCDDA content_{HPLC}: 46.06% by wt.

Content of compounds of the formula 36: 52.45% by wt.

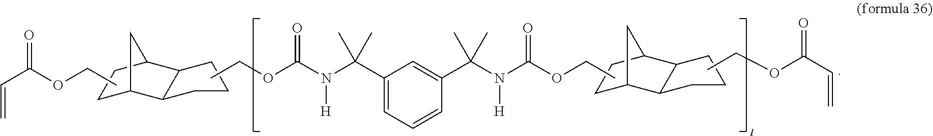

### Example 6

Synthesis of Free-Radically Polymerizable Compounds with H6XDI

To an initial charge of 22 g of oil from example 1 in 15 ml of THF in a 100 ml two-neck flask with magnetic stirrer bar, internal thermometer and dropping funnel with a CaCl₂ drying tube was slowly added dropwise, at RT, 3.89 g of H6XDI in 15 ml of THF. 10 μl of catalyst solution (DMTND in toluene-t, weight ratio 1:1) was added and the mixture was stirred at 40° C. for 5 hours and at RT for 18 hours. Subsequently, isocyanate groups were no longer detectable by FT-IR spectroscopy.

The reaction mixture was transferred to a 100 ml one-neck flask, a spatula-tip of BHT was added, and THF was removed as far as possible on a rotary evaporator. Further volatile constituents were removed under fine vacuum. What remained was a clear, colorless oil.

Yield: 21.48 g

Drying loss (2 hours, 110° C.): 1.43%

By HPLC-MS, TCDDA was detectable at a retention time of 34.0 min, and the product (M+H⁺=696) at a retention time of 43.3 min.

GPC: TCDDA had an elution volume of 29.7 ml, and the product elution volumes of 26.6 ml, 25.1 ml, 24.1 ml and 23.4 ml.

It will be apparent that different isomeric structures arise from the isomers or isomer mixtures of the isocyanates and/or alcohols used in the syntheses of the free-radically polymerizable compounds, in particular with regard to the di-methylenetricyclodecane groups and their direction in the polymerizable compounds, for example in an oligomer chain. However, the single compounds of the structures of the formulae 33 to 34 shown here are each intended to include all the resulting isomeric compounds.

### 5. Properties of the Free-Radically Polymerizable Compositions

For production of the compositions, the individual components were mixed by magnetic stirrer until the composition was homogeneous.

TABLE 2
Composition, viscosity (η), flexural strength (FS), modulus of elasticity
(ME), conversion (C), volume shrinkage (VS), refractive index uncured (n_{D}VA) and
refractive index cured (n_{D}NA) of compositions of the invention (ex. A, B,
C, D) containing the polymerizable compounds free-radically synthesized in
examples 2-5 (oil from Ex. 2-5) and of comparative compositions (CE A, B)

| Component | | | | | | |
|---|---|---|---|---|---|---|
| [% by wt.] | Ex. A | Ex. B | Ex. C | Ex. D | CE A | CE B |
| Ex. 2 | 97.4 | | | | | |
| Ex. 3 | | 97.4 | | | | |
| Ex. 4 | | | 97.4 | | | |
| Ex. 5 | | | | 97.4 | | |
| Bis-GMA | | | | | 68.2 | 48.7 |
| TEDMA | | | | | 29.2 | 48.7 |
| CQ | 1.0 | 1.0 | 1.0 | 1.0 | 1.0 | 1.0 |
| EHA | 1.598 | 1.598 | 1.598 | 1.598 | 1.598 | 1.598 |
| BHT | | 0.002 | | 0.002 | 0.002 | 0.002 |
| η [Pa · s] | 18.8 | 13.8 | 16.6 | 20.6 | | |
| FS [MPa] | | 100 ± 7 | | 129 ± 8 | 105 ± 8 | 96 ± 7 |
| ME [GPa] | | 2.6 ± 0.1 | | 3.3 ± 0.1 | 2.6 ± 0.1 | 2.4 ± 0.2 |
| C [%] | | 72.0 | | 74.4 | 68.3 | — |
| VS [%] | | 4.4 ± 0.3 | | 4.6 ± 0.2 | 6.4 ± 0.3 | 8.2 ± 0.3 |
| n_{D}VA | 1.5144 | 1.5157 | 1.5121 | 1.5198 | 1.5214 | 1.5046 |
| n_{D}NA | | 1.5425 | | 1.5463 | 1.5529 | 1.5388 |

TABLE 3
Composition, flexural strength (FS), modulus of elasticity (ME),
volume shrinkage (VS), refractive index unhardened (n_{D}VA), refractive
index hardened (n_{D}NA) of comparative compositions (CE C-F)

| Component [% | | | | |
|---|---|---|---|---|
| by wt.] | CE C | CE D | CE E | CE F |
| UDMA | 77.9 | 77.9 | 53.6 | 53.6 |
| TCDDA | | | 43.8 | |
| TCDDMA | | 19.5 | | |
| TEDMA | 19.5 | | | 43.8 |
| CQ | 1.0 | 1.0 | 1.0 | 1.0 |
| EHA | 1.598 | 1.598 | 1.598 | 1.598 |
| BHT | 0.002 | 0.002 | 0.002 | 0.002 |
| FS [MPa] | 102 ± 4 | 94 ± 6 | — | — |
| ME [GPa] | 2.4 ± 0.1 | 2.5 ± 0.1 | — | — |
| VS [%] | 7.0 ± 0.6 | 6.9 ± 0.3 | — | — |
| n_{D}VA | 1.479 | 1.488 | 1.4940 | 1.4749 |
| n_{D}NA | 1.5100 | 1.5215 | 1.5170 | 1.5108 |

Inventive examples B and D show significantly lower volume shrinkage than comparative examples A-F. The conversions, flexural strengths and modulus of elasticity of the inventive examples are in the region of comparative examples A-F and in some cases higher.

The difference between the refractive indices of the cured inventive examples B and D and the dental glass used (n_{D}=1.53) is comparable to the difference between comparative examples A-F and the same dental glass. The result for inventive examples B and D is thus similarly low turbidity of the dental composites, which lead to very good esthetic product properties.

### Production of Dental Composites

For production of the dental composites, the components (oil ex. 3, ex. 5, and bisGMA and TEDMA) were first mixed with CQ, EHA and BHT by magnetic stirrer until the composition was homogeneous. Subsequently, a total of 75% by weight of BaF, based on the total mass of the dental composite, was added gradually, and the mixture was homogenized by means of a Speedmixer DAC 400-1 VAC-P (from Hauschild, Germany) and degassed at 20 mbar for 3 min. The compositions and measurement results are given in table 4.

TABLE 4
Composition, flexural strength (FS), modulus of elasticity
(ME), conversion (C) and volume shrinkage (VS) of dental
composites (ex. E, F) containing the polymerizable compounds
synthesized free-radically in Examples 3 and 5 (ex. 3,
ex. 5) and the comparative composition G (CE G)

| Component | | | |
|---|---|---|---|
| [% by wt.] | Ex. E | Ex. F | CE G |
| Ex. 3 | 24.8325 | | |
| Ex. 5 | | 24.8325 | |
| Bis-GMA | | | 12.41625 |
| TEDMA | | | 12.41625 |
| CQ | 0.0625 | 0.0625 | 0.0625 |
| EHA | 0.1045 | 0.1045 | 0.1045 |
| BHT | 0.0005 | 0.0005 | 0.0005 |
| BaF | 75 | 75 | 75 |
| Total | 100 | 100 | 100 |
| FS [MPa] | 133 ± 6 | 134 ± 6 | 128 ± 16 |
| ME [GPa] | 9.4 ± 0.4 | 10.3 ± 0.2 | 9.9 ± 0.2 |
| C [%] | 65.0 | 65.9 | 73.2 |
| VS [%] | 1.9 ± 0.3 | 2.1 ± 0.1 | 3.6 ± 0.3 |

Examples E and F, in which the compounds of the invention were used, show distinctly lower volume shrinkage than comparative example G, in which a monomer mixture from the prior art has been used. Conversions, flexural strengths and the moduli of elasticity of the inventive examples are comparable to comparative example G.

For comparison, the properties of commercially available dental composites with a higher filler level that are known in the prior art are shown in table 5. In spite of a higher filler level, the shrinkage data for these composites is higher than for the inventive examples.

TABLE 5
Properties of prior art, commercially available dental
composites containing BPA-containing polymerizable
compositions and 78.5% to 83% by weight filler.

| Dental composite | FS [MPa] | |
|---|---|---|
| (batch) | ME [GPa] | VS [%] |
| Ecosite Elements A2 | 135 ± 9 | 2.5 ± 0.1 |
| (#798872) | 10.7 ± 0.8 | |
| Filtek Supreme XTE | 149 ± 15 | 2.2 ± 0.0 |
| Dentin A2 (#N902487) | 11.7 ± 0.4 | |
| Tetric EvoCeram A2 | 118 ± 5 | 2.3 ± 0.2 |
| (#W97572) | 9.2 ± 0.9 | |

1. A free-radically polymerizable compound, represented by a structure of the formula 1: 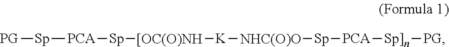
where
PG=each independently selected from a free-radically polymerizable group,
R¹=selected from hydrogen, a C1-C8-alkyl group, an aryl group and an araliphatic group with C6-C8 carbon atoms,
R²=selected from hydrogen, a C1-C4-alkyl group;
Sp=a spacer group selected independently from unbranched and branched alkylene having C1-C19 carbon atoms, which may additionally contain oxygen, sulfur and/or —OOC— in the carbon chain,
or Sp is absent;
PCA=each independently a polycyclic group;
K=an aliphatic acyclic, saturated or unsaturated unit having C1-C15 carbon atoms, where the unit may be substituted by one or more C1-C3 aliphatic carbon substituents,
an aliphatic cyclic, saturated or unsaturated unit having C3-C15 carbon atoms, where the unit may be substituted by one or more aliphatic C1-C3 carbon substituents, or
an aromatic or araliphatic unit having C6-C14 carbon atoms, where the unit may be substituted by one or more aliphatic C1-C3 carbon substituents;
and
n=1 to 9.

2. A free-radically polymerizable compound as claimed in claim 1, characterized in that the spacer group Sp is selected from methylene, *CH₂—(OC₂H₄)ₚ, *CH₂—(OC₃H₆)ₚ, *(OC₂H₄)ₚ, *(OC₃H₆)ₚ, *CH₂—(O—C(O)—R⁵-)p, *(O—C(O)—R⁵-)p and *S—R⁵, where p=1-5, and R⁵ is a C1-C12 alkylene group.

3. A free-radically polymerizable compound as claimed in claim 1, characterized in that the polycyclic group PCA is selected in each case from structures of the formulae 2 to 13: 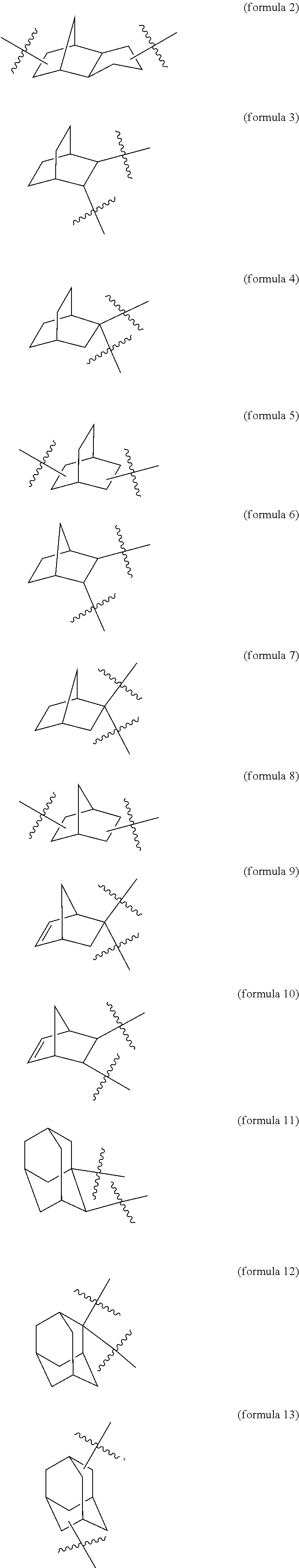
which may optionally be substituted by one or more C1-C4 alkyl groups.

4. A free-radically polymerizable compound as claimed in claim 1, characterized in that the polycyclic group PCA is an aliphatic tricyclic group.

5. A free-radically polymerizable compound as claimed in claim 1, characterized in that K is selected from the following structures of the formulae 14 to 26:

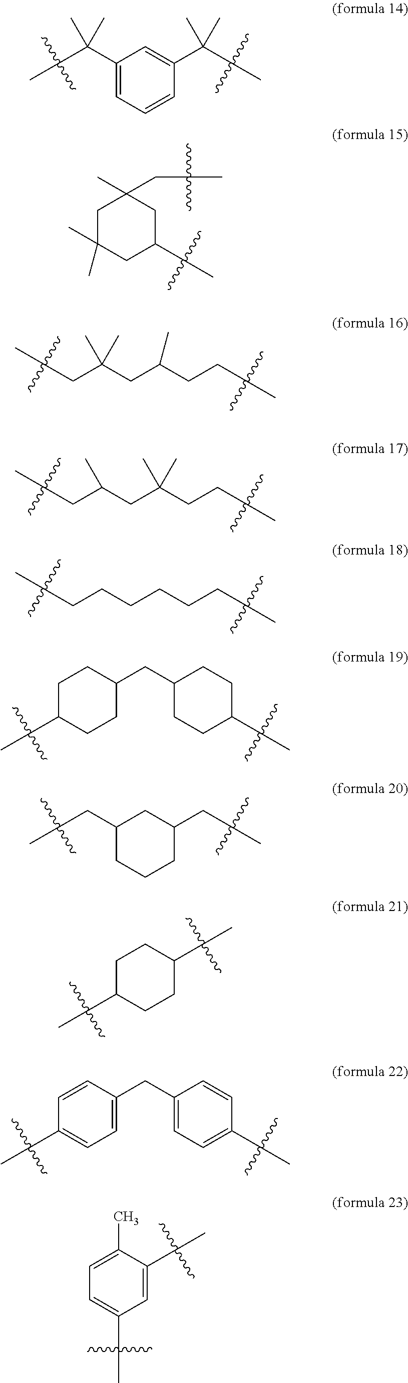

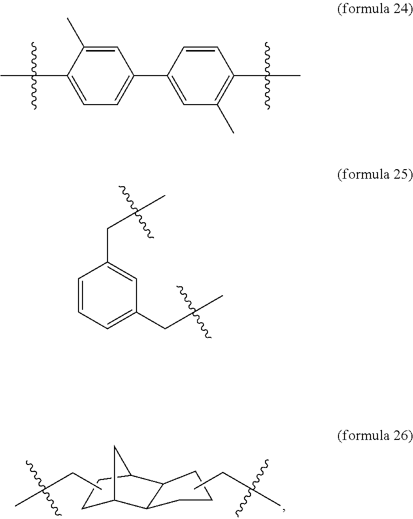

6. A free-radically polymerizable compound as claimed in claim 1, characterized in that the free-radically polymerizable compound is represented by a structure of the formula 27: 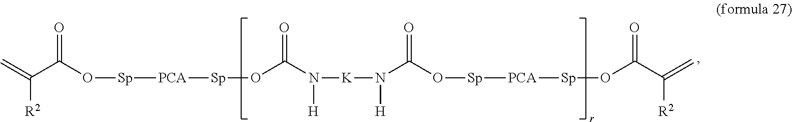
where
R²=hydrogen or a methyl group;
Sp=a spacer group selected independently from unbranched and branched alkylene having C1-C19 carbon atoms, which may additionally contain oxygen, sulfur and/or —OOC— in the carbon chain,
or Sp is absent;
PCA=each independently a polycyclic group;
K=an aliphatic acyclic, saturated or unsaturated unit having C1-C15 carbon atoms, where the unit may be substituted by one or more C1-C3 aliphatic carbon substituents,
an aliphatic cyclic, saturated or unsaturated unit having C3-C15 carbon atoms, where the unit may be substituted by one or more aliphatic C1-C3 carbon substituents, or
an aromatic or araliphatic unit having C6-C14 carbon atoms, where the unit may be substituted by one or more aliphatic C1-C3 carbon substituents;
r=1 to 9.

7. A free-radically polymerizable compound as claimed in claim 1, characterized in that the free-radically polymerizable compound is represented by a structure selected from the formulae 28-31: 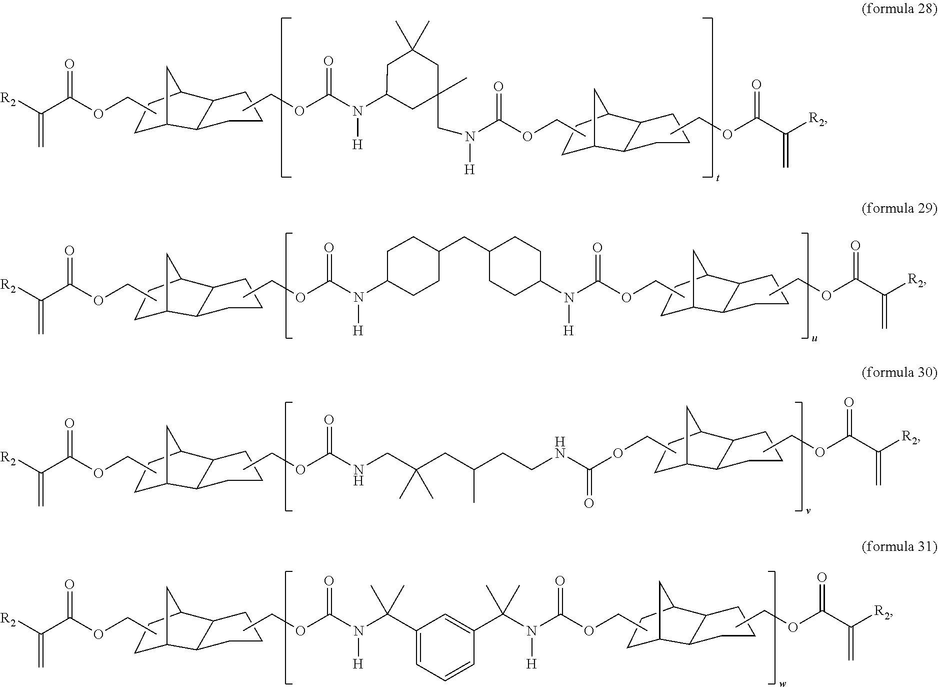
where
t, u, v and w=each independently 1-9; and
R²=selected from hydrogen, a C1-C4 alkyl group.

8. A process for preparing a free-radically polymerizable compound, characterized in that the process comprises the following steps:
a) reacting a diol of a polycyclic group PCA with a (meth)acrylic ester to give a PCA mono(meth)acrylate having a hydroxyl group by enzymatic catalysis,
b) reacting the PCA mono(meth)acrylate containing the hydroxyl group from step a) with a diisocyanate compound.

9. A free-radically polymerizable composition comprising:
a) one or more free-radically polymerizable compounds as claimed in claim 1;
b) optionally one or more further free-radically polymerizable monomers or oligomers not covered by the at least one compound of the formula 1;
c) optionally at least one initiator or initiator system for the polymerization;
d) optionally one or more stabilizers;
e) optionally one or more fillers;
f) optionally customary dental additives.

10. The free-radically polymerizable composition as claimed in claim 9, characterized in that the free-radically polymerizable composition includes one or more free-radically polymerizable compounds of the formula 1 in a proportion by mass of 1-100% by weight, based on the total mass of all free-radically polymerizable monomers and oligomers in the polymerizable composition.

11. The free-radically polymerizable composition as claimed in claim 9, characterized in that multiple compounds of the formula 1, selected from monomers (n=1) and/or oligomers (n=2-9), are present.

12. The free-radically polymerizable composition as claimed in claim 9, characterized in that the free-radically polymerizable composition is a free-radically polymerizable dental material.

13. A polymerizable dental material comprising the free-radically polymerizable composition of claim 9, wherein the polymerizable dental material is a dental composite, dental cement, self-adhesive dental cement, dental lacquer, core build-up material, root canal filling material, filling material, lining material, luting material, crown material, bridge material, restoration material, orthodontic material and/or prosthesis material.

14. A method of producing a material selected from orthodontic materials, aligners, rail materials, prosthesis base materials, modeling materials, crown and bridge materials, drilling templates, gingiva masks, spoon materials, mouth guards or veneers, comprising 3D printing the polymerizable dental material of claim 9.

15. A cured dental material produced from a polymerizable composition as claimed in claim 9.

16. A free-radically polymerizable compound as claimed in claim 1, wherein:
PG=OOC—CR²═CH₂,
R¹=selected from a C1-C8-alkyl group and a benzyl group,
R²=selected from hydrogen and methyl;
PCA=an aliphatic bi- or tricyclic group;
K=an aliphatic acyclic, saturated or unsaturated unit having C6-C9 carbon atoms, where the unit may be substituted by one or more C1-C3 aliphatic carbon substituents,
an aliphatic cyclic, saturated or unsaturated unit having C6-C13 carbon atoms, where the unit may be substituted by one or more aliphatic C1-C3 carbon substituents, or
an aromatic or araliphatic unit having C6-C13 carbon atoms, where the unit may be substituted by one or more aliphatic C1-C3 carbon substituents;
n=1 to 6.

17. A free-radically polymerizable compound as claimed in claim 4, characterized in that the polycyclic group PCA is tricyclo[5.2.1.0/2,6]decanylene (TCD) (formula 2).

18. A free-radically polymerizable compound as claimed in claim 5, characterized in that K is selected from the structures of formulae 14, 15, 16, 17, 18, 19 and 20.

19. A free-radically polymerizable compound as claimed in claim 6, characterized in that the free-radically polymerizable compound is represented by a structure of the formula 27: 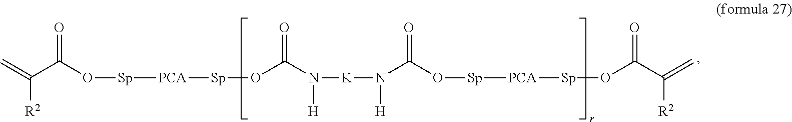
where
PCA=tricyclo[5.2.1.0/2,6]decanylene;
K=an aliphatic acyclic, saturated or unsaturated unit having C6-C9 carbon atoms, where the unit may be substituted by one or more C1-C3 aliphatic carbon substituents,
an aliphatic cyclic, saturated or unsaturated unit having C6-C13 carbon atoms, where the unit may be substituted by one or more aliphatic C1-C3 carbon substituents, or
an aromatic or araliphatic unit having C6-C13 carbon atoms, where the unit may be substituted by one or more aliphatic C1-C3 carbon substituents;
r=1 to 6.

20. A free-radically polymerizable composition as claimed in claim 10, characterized in that the free-radically polymerizable composition includes one or more free-radically polymerizable compounds of the formula 1 in a proportion by mass of 10-65% by weight, based on the total mass of all free-radically polymerizable monomers and oligomers in the polymerizable composition.
